(19) United States
(12) Patent Application Publication
(10) Pub. No.: US 2020/0261558 A1
(43) Pub. Date: Aug. 20, 2020

(54) Vaccines Against Leishmania Infection

(71) Applicant: Georgia Tech Research Corporation, Atlanta, GA (US)

(72) Inventors: M.G. Finn, Atlanta, GA (US); Alexandre F. Marques, Atlanta, GA (US)

(21) Appl. No.: 16/639,300

(22) PCT Filed: Aug. 15, 2018
(86) PCT No.: PCT/US18/00134

§ 371 Date: Feb. 14, 2020

Related U.S. Application Data
(60) Provisional application No. 62/545,813, filed on Aug. 15, 2017.

(51) Int. Cl. A61K 39/008; A61K 9/51

(52) U.S. Cl. CPC A61K 39/008; A61K 9/51

(57) ABSTRACT

A vaccine composition for treating and preventing infection by protozoans is disclosed. The vaccine composition comprises carbohydrates and/or peptides present on the surface of the protozoan, optionally bound to an immunogenic protein nanoparticle.

### CROSS-REFERENCE TO RELATED APPLICATIONS

This application claims priority to U.S. Provisional Application No. 62/545,813, filed on Aug. 15, 2017, the disclosure of which is herein incorporated by reference in its entirety.

### GOVERNMENT SPONSORSHIP

This invention was made with government support under Grant No. 101421 (R01 GM101421, 3306JR5) awarded by the National Institutes of Health. The government has certain rights in the invention.

### BACKGROUND OF THE INVENTION

### 1. Field of the Invention

The invention relates to the field of vaccines and to the use of carbohydrates and peptide-based molecules to stimulate an immune response against a protozoan parasite of the Leishmania genus (e.g., Leishmania infantum and Leishmania amazonensis). Provided herein are antigenic carbohydrates and peptide-based molecules as well as immunogenic compositions bearing these antigenic carbohydrates and peptide-based molecules, such as protein nanoparticles and virus-like particles. Also provided herein are methods of inducing an immune response and treating a subject infected with a protozoan parasite of the Leishmania genus.

### 2. Background

Leishmaniasis, a major human health problem endemic in more than 88 countries, is spread by sand flies, which regurgitate the parasite into the skin of their vertebrate hosts (1). Leishmania species cause a spectrum of clinical symptoms (2,3) depending on the nature of the infecting strain and its interaction with the host immune system and other genetic factors (4). In Mediterranean and South American countries, cutaneous leishmaniasis (CL) in humans is caused primarily by L. mexicana, L. amazonensis, and L. braziliensis, all of which induce ulcers localized to the skin. L. amazonensis is also responsible for diffuse cutaneous leishmaniasis, whereas L. braziliensis, in some circumstances, may cause lesions in mucosal surfaces. Leishmania infantum (in Europe, North Africa, and Latin America) and Leishmania donovani (in East Africa and the Indian subcontinent) are the causative agents of visceral leishmaniasis (VL) (5), in which the parasite spreads to internal organs (primarily the liver, spleen and bone marrow) and can be lethal if not treated (6,7).

The carbohydrate-rich molecules found on the surface of Leishmania parasites include lipophosphoglycans, glycosylphosphatidylinositol-anchored proteins, glycosylphosphatidylinositol lipids, and proteophosphoglycans (8). These glycoproteins are part of the glycocalyx of the promastigote infective form, playing an important role in infectivity (9,10) and sand fly interaction (11). After transmission by blood-feeding sand flies of the genus Lutzomyia (in the New World), metacyclic Leishmania parasites interact primarily with phagocytic cells and then may be distributed to their resident tissues and organs, depending of the Leishmania species. While carbohydrates present on the parasite surface are likely to play an important role in mediating cell host adhesion (12), the molecular mechanism of virulence of this parasitic disease is poorly understood.

Among the most interesting pathogen-related carbohydrates is the α-Gal epitope, referring to both the Gal-α1,3-Gal disaccharide (13) and the Gal-α1,3-Gal-β1,4-GlcNAc trisaccharide (14).

The human immune system recognizes and reacts against the α-Gal epitope as a marker of infection. Due to the inactivation of the α1,3-galactosyltransferase (α1,3GalT) gene terminal, non-reducing and linear α-Gal epitopes are absent and, therefore, highly immunogenic to humans. For example, healthy human individuals normally produce anti-α-Gal antibodies (also known as normal anti-Gal antibodies) against α-Gal epitopes found in lipopolysaccharides of enterobacteria. During infections with Trypanosoma cruzi, Leishmania spp., or Plasmodium spp., parasite-specific anti-α-Gal antibodies are elicited. These antibodies have different specificities than normal anti-α-Gal antibodies and are specific to the parasites that elicited them. Further, approximately 1-2% of circulating antibodies in healthy humans recognize α-Gal epitopes, but these antibodies are mostly of the IgE and IgM subtypes.

There is a need for a novel therapeutic vaccine against a protozoan parasite of the Leishmania genus that takes advantage of the immune response to certain carbohydrates and/or peptide-based molecules present on and/or associated with the protozoan.

### BRIEF SUMMARY OF THE INVENTION

As specified in the Background Section, there is a great need in the art to identify technologies for protozoan parasite vaccines, including vaccines for protozoans of the Leishmania genus. The inventors have developed a novel therapeutic vaccine against protozoans of the Leishmania genus that takes advantage of the immune response to certain carbohydrates and/or peptide-based molecules present on and/or associated with the protozoan. The therapeutic vaccine and methods described herein may be useful to immunize against species of Leishmania, such as, but not limited to, L. mexicana, L. major, L. tropica, L. amazonensis, L. infantum, L. donovani and L. braziliensis, that also have the immunogenic carbohydrate.

In one aspect, the invention provides a vaccine comprising at least one carbohydrate and/or peptide-based molecule associated with a Leishmania protozoan, such as Leishmania infantum and Leishmania amazonensis. In some embodiments, the vaccine comprises at least one carbohydrate selected from the group consisting of the Gal-α1,3-Galβ disaccharide and the Gal-α1,3-Gal-β1,4-GlcNAc trisaccharide (herein referred to as the “α-Gal epitope”). In some embodiments, the carbohydrate is associated with an immunogenic protein nanoparticle. In some embodiments, the immunogenic protein nanoparticle is a virus-like particle. In certain embodiments, the virus-like particle is Qβ or PP7.

In some embodiments, the vaccine comprises at least one peptide-based molecule, wherein the peptide-based molecule is

In another aspect, the invention provides a method of inducing an immune response in a subject against a Leishmania protozoan, such as Leishmania infantum and Leishmania amazonensis, the method comprising administering to the subject at least one of the vaccines of the present invention. In one embodiment, the subject is currently infected with Leishmania and the vaccine induces an immune response against Leishmania. In one embodiment, the subject is not currently infected with Leishmania and the vaccine induces an immune response against Leishmania. In one embodiment, the subject is currently infected with Leishmania infantum and the vaccine induces an immune response against Leishmania infantum. In one embodiment, the subject is not currently infected with Leishmania infantum and the vaccine induces an immune response against Leishmania infantum. In one embodiment, the subject is currently infected with Leishmania amazonensis and the vaccine induces an immune response against Leishmania amazonensis. In one embodiment, the subject is not currently infected with Leishmania amazonensis and the vaccine induces an immune response against Leishmania amazonensis. In one embodiment, the subject has cutaneous, visceral, and/or mucocutaneous leishmaniasis, and the vaccine induces an immune response against Leishmania.

In another aspect, the invention provides a method of treating a subject infected with a protozoan parasite of the Leishmania genus (e.g., L. mexicana, L. major, L. tropica, L. amazonensis, L. infantum, L. donovani and L. braziliensis), the method comprising administering to the subject at least one of the vaccines of the present invention. In one embodiment, the method further comprises the administration of one or more of an antibiotic, such as an anti-fungal drug, an anti-viral drug, an anti-parasitic drug, an anti-protozoal drug, and an anti-helminthic drug.

In another aspect, the invention provides a method of treating a subject infected with a protozoan parasite of the Leishmania genus (e.g., L. mexicana, L. major, L. tropica, L. amazonensis, L. infantum, L. donovani and L. braziliensis), the method comprising administering to the subject at least one antibody, wherein the at least one antibody specifically binds to at least one of the immunogenic carbohydrates of the present invention. In one embodiment, the method further comprises the administration of an antibiotic, such as an anti-fungal drug, an anti-viral drug, an anti-parasitic drug, an anti-protozoal drug, an anti-helminthic drug, or a combination thereof.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 depicts a schematic of the α-Gal trisaccharide epitope on the surface of a protein nanoparticle and on the surface of the protozoan parasite Leishmania.

FIGS. 2A-2F depict α-Gal epitope identification on the surface of Leishmania protozoans. (2A, 2B) Fluorescence microscopy of L. amazonensis and L. infantum promastigote cells labeled with 3-5 μg/mL of FITC-labeled MOA (grey=labeled MOA, white arrows=DAPI-stained nuclei, scale bar=10 μm). (2C-2D) Flow cytometry (FACScan) of the cells treated as in FIGS. 2A and 2B; x-axis=fluorescein fluorescence (488 nm excitation). “+ Gal” represents the same samples pretreated with 0.2 M galactose before addition of MOA-FITC. (2E) ELISA performed with the indicated Leishmania extracts (10 μg/mL) as antigen, and biotinylated MOA (2 μg/mL) and anti-α-Gal antibody (3 μg/mL) as probe reagents. Qβ virus-like particles (2 μg/mL) bearing high density of either α-Gal or glucose were used as positive and negative controls, respectively. (2F) ELISA as in FIG. 2E, with pretreatment of the antigens with green coffee bean α-galactosidase (overnight, 10 U/mL). All experiments were performed in triplicate.

FIGS. 3A-3F show infection with Leishmania parasites. (3A) Anti-α-Gal serum IgG antibody levels in infected α-galactosyltransferase wild type mice (αGalT-WT, serum dilution 1/100). (3B) Anti-α-Gal serum IgG antibody levels in infected α-galactosyltransferase knockout mice (αGalT-KO, serum dilution 1/100); same key as FIG. 3A. (3C, 3E) Parasite load levels (qPCR) in liver and spleen samples of αGalT-WT and αGalT-KO mice infected with 10⁷ Leishmania amazonensis cells. (3D, 3F) Parasite load levels (qPCR) in liver and spleen samples of αGalT-WT and αGalT-KO infected mice with 10⁷ Leishmania infantum cells. Three or more independent experiments were performed in each group containing 3-7 mice.

FIGS. 4A-4E show anti-α-Gal IgG antibody production by mice vaccinated with Qβ-α-Gal antigen. (4A) Protocol employing 13 mice per group. (4B-4E) ELISA for detection of serum antibodies (serum dilution 1/100); x-axis=each mouse in the group, arbitrarily numbered. (4B) Serum from immunized mice, against Qβ-α-Gal immobilized on the ELISA plate; (4C) serum from nave mice, against plated Qβ-α-Gal; (4D) serum from immunized mice, against plated Qβ-Glc; (4) serum from nave mice, against plated Qβ-Glc.

FIGS. 5A-5D show protection from Leishmania infection in α-GalT-KO mice. (5A) Vaccination and infection protocol. (5B) Detection of anti-α-Gal IgG antibody response by ELISA at week 2.5, for animals immunized with Qβ-α-Gal vs Qβ-Glc. “L.a.”=mice later infected with L. amazonensis; “L.i.”=mice later infected with L. infantum. (5C) qPCR determination of parasite load from liver and spleen of vaccinated and unvaccinated αGalT-KO mice infected subcutaneously with 10⁷ promastigotes of Leishmania amazonensis. (5D) qPCR determination of parasite load from liver and spleen of vaccinated and unvaccinated αGalT-KO mice infected intraperitoneally with 10⁷ promastigotes of Leishmania infantum. Three or more independent experiments were performed in groups containing from 3 to 7 mice.

### DETAILED DESCRIPTION OF THE INVENTION

As specified in the Background Section, there is a great need in the art to identify technologies for Leishmania protozoan vaccination and therapeutics, such as L. mexicana, L. major, L. tropica, L. amazonensis, L. infantum, L. donovani and L. braziliensis protozoan vaccination and therapeutics.

Secreted and surface-displayed carbohydrates are essential for virulence and viability of many parasites, including for immune system evasion. The inventors have identified the α-Gal trisaccharide epitope on the surface of the protozoan parasites Leishmania infantum and Leishmania amazonensis, the etiological agents of visceral and cutaneous leishmaniasis, respectively, with the latter bearing larger amounts of α-Gal than the former. A polyvalent α-Gal conjugate on the immunogenic Qβ virus-like particle was tested as a vaccine against Leishmania infection in a C57BL/6 α-galactosyltransferase knockout mouse model, which mimics human hosts in producing high titers of anti-α-Gal antibodies. α-Gal-T knockout mice infected with promastigotes of both Leishmania species showed significantly lower parasite load in the liver and slightly decreased levels in the spleen, compared with wild-type mice. Vaccination with Qβ-α-Gal nanoparticles protected the knockout mice against Leishmania challenge, eliminating the infection and proliferation of parasites in the liver and spleen as probed by qPCR. The α-Gal epitope may therefore be considered as a vaccine candidate to block human cutaneous and visceral leishmaniasis.

As shown herein, the α-Gal epitope has been identified on the promastigote form of Leishmania amazonensis and Leishmania infantum. Also shown herein is the use of the α-galactosyltransferase knockout mouse (αGalT-KO) of Thall and colleagues (19) as a murine model for leishmaniasis, and a polyvalent α-Gal conjugate as an effective vaccine candidate against parasite infection in this model. Since only humans and higher apes among all mammals lack the α-galactosyltransferase gene (20), the αGalT-KO mouse mimics the human immune system's ability to generate anti-α-Gal antibodies to a much greater degree than wild-type rodents. These mice have been used primarily in studies of xenotransplant rejection and fertilization (21,22), but are also of great potential interest for studies of any pathogen that displays α-Gal.

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

“About” as used herein when referring to a measurable value such as an amount, a temporal duration, and the like, is meant to encompass variations of ±20%, ±10%, ±5%, ±1%, or ±0.1% from the specified value.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a vaccine or a composition according to the invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The term “antibody,” as used herein, refers to an immunoglobulin molecule which specifically binds with an antigen. An antibody can be an intact immunoglobulin derived from a natural source or from a recombinant source. Such antibody can comprise an immunoreactive portion of an intact immunoglobulin. The antibody may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. Any macromolecule, including virtually all proteins or peptides, can serve as an antigen. Furthermore, antigens can be derived from recombinant or genomic DNA. Any DNA which comprises a nucleotide sequences or a partial nucleotide sequence encoding a protein that elicits an immune response therefore encodes an “antigen” as that term is used herein. Furthermore, an antigen need not be encoded solely by a full-length nucleotide sequence of a gene. Partial nucleotide sequences of more than one gene may be used, for example these nucleotide sequences may be arranged in various combinations to elicit a desired immune response. Moreover, an antigen need not be encoded by a “gene” at all. An antigen can be generated synthesized or can be derived from a biological sample.

The term “agent” includes any substance, metabolite, molecule, element, compound, or a combination thereof. It includes, but is not limited to, e.g., protein, oligopeptide, small organic molecule, glycan, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

The term “binding” refers to a direct association between at least two molecules, due to, for example, covalent, electrostatic, hydrophobic, ionic and/or hydrogen-bond interactions.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. The acceptor antibody heavy and light chains are not required to originate from the same acceptor antibody.

The term “immunoglobulin” or “Ig,” as used herein, is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing release of mediators from mast cells and basophils upon exposure to allergen.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

In the context of the present invention, the term “vaccine” (also referred to as an immunogenic composition) refers to a substance that induces anti-Leishmania protozoan immunity or suppresses leishmaniasis (e.g., cutaneous, visceral, and mucocutaneous leishmaniasis) upon inoculation into an animal.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Throughout this disclosure, various aspects of the invention can be presented in a range format. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

The invention provides an immunological composition comprising a carbohydrate, peptide, peptide-based molecule or combination of peptide-based molecules derived from carbohydrates and/or proteins associated with Leishmania protozoans (e.g., L. mexicana, L. major, L. tropica, L. amazonensis, L. infantum, L. donovani and L. braziliensis protozoans), useful in eliciting an immune response. The compositions comprising one or more carbohydrates and/or peptide-based molecules of the invention not only are useful as a prophylactic therapeutic agent for immunoprotection but are also useful as a therapeutic agent for treatment of an ongoing disease or disorder associated with infection by Leishmania protozoans (e.g., L. mexicana, L. major, L. tropica, L. amazonensis, L. infantum, L. donovani and L. braziliensis protozoans) in a subject, including but not limited to cutaneous, visceral, and mucocutaneous leishmaniasis.

The invention relates to the administration of at least one carbohydrate, peptide, and/or peptide-based molecule associated with Leishmania protozoans (e.g., L. mexicana, L. major, L. tropica, L. amazonensis, L. infantum, L. donovani and L. braziliensis protozoans), to a subject to induce an immune response. Thus, the present invention provides a carbohydrate, a peptide or a combination of peptides, a peptide-based molecule or combination of peptide-based molecules, or a polynucleotide or a combination of polynucleotides, which are useful in inducing an immune response, for the treatment or prevention of infection by Leishmania protozoans (e.g., L. mexicana, L. major, L. tropica, L. amazonensis, L. infantum, L. donovani and L. braziliensis protozoans), including but not limited to cutaneous, visceral, and mucocutaneous leishmaniasis.

In one embodiment, the composition of the invention comprises a carbohydrate, or fragments or variants thereof, associated with Leishmania protozoans (e.g., L. mexicana, L. major, L. tropica, L. amazonensis, L. infantum, L. donovani and L. braziliensis protozoans). In various embodiments, the carbohydrate comprises one or both of the Gal-α1,3-Galβ disaccharide and the Gal-α1,3-Gal-β1,4-GlcNAc trisaccharide (herein referred to as the “α-Gal epitope”), and fragments or variants thereof.

In a particular embodiment, the composition of the invention comprises the α-Gal epitope, or a fragment or variant thereof. At least one carbohydrate associated with Leishmania protozoans (e.g., L. mexicana, L. major, L. tropica, L. amazonensis, L. infantum, L. donovani and L. braziliensis protozoans), useful in eliciting an immune response, can be used alone or in any combination for eliciting an immune response.

In one embodiment, the composition of the invention comprises a peptide or peptide-based molecule associated with Leishmania protozoans (e.g., L. mexicana, L. major, L. tropica, L. amazonensis, L. infantum, L. donovani and L. braziliensis protozoans). Four classes of peptides or peptide-based molecules may be used in compositions of the invention. The first class comprises peptides that are derivatized with glycans expressed by the Leishmania protozoan; also referred to as “glycopeptides.” The peptide sequences may or may not correspond to sequences expressed by the parasite. The compositions of the invention can comprise different glycopeptides, including glycopeptides that differ in their peptide sequence and/or glycan structure. The second class comprises peptides of 6 to 50 amino acids in length, of sequences selected from proteins expressed by the Leishmania protozoan, including peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length. More than one such peptide may be used in compositions of the invention. The third class comprises protein domains (generally larger than 50 amino acids) and/or whole proteins expressed by the Leishmania protozoan, and combinations thereof. The fourth class comprises peptides of length 6-50 amino acids in length that are not found in proteins produced by Leishmania protozoans, but that have three-dimensional structures which mimic peptide or glycan structures produced by the protozoan. Such mimicking peptides include peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length. More than one such peptide may be used in compositions of the invention. Combinations of any or all of these four classes may also be included in compositions of the invention.

In one embodiment, the composition of the invention comprises a carbohydrate, peptide, peptide-based molecule or combination thereof derived from carbohydrates and/or proteins associated with Leishmania protozoans, useful in eliciting an immune response. Non-limiting examples of carbohydrates can comprise carbohydrates (such as α-Gal epitopes) attached by a variety of linkages to proteins such as keyhole limpet hemocyanin, serum albumin, tetanus toxoid, or virus-like particles. Furthermore, the point of attachment can be to a specific amino acid residue in a defined peptide sequence that either mimics the sequence of a peptide expressed by the Leishmania protozoan or is displayed effectively by the immune system, such as so-called class-I or class-II peptides (referring to sequences that bind well to major histocompatibility complex proteins class I or class II). The linkage between the carbohydrate and peptide/protein can be both “natural” (O-linked to serine or threonine, N-linked to asparagine) and “unnatural.” Unnatural connections are made by a reaction between a functional group installed on a tether installed at the reducing end of the carbohydrate and a complementary functional group installed on the peptide or protein. Such groups can be those that participate in bioconjugation reactions such as, but not limited to, the following: azides and alkynes (cycloaddition), dienes and dienophiles (Diels-Alder reaction), activated esters and amines (peptide bond formation), maleimides and thiols (thiol alkylation), tetrazines and activated alkenes (tetrazine ligation), aldehydes and aminoethers, hydrazines, or hydrazones (oxime or hydrazone formation). The linkers installed on the carbohydrate and/or peptide/protein can be of chain lengths from 1 to 100 atoms, and be composed of alkyl chains, aryl groups, peptides, oligo(ethylene glycol) chains, peptoids, oligo- or polyesters, oligo- or polyacrylates, and/or oligo- or polyamides.

The present invention also provides methods of preventing, inhibiting, and treating infection by Leishmania protozoans (e.g., L. mexicana, L. major, L. tropica, L. amazonensis, L. infantum, L. donovani and L. braziliensis protozoans) in a subject in need thereof. In one embodiment, the methods of the invention induce immunity against Leishmania protozoans (e.g., L. mexicana, L. major, L. tropica, L. amazonensis, L. infantum, L. donovani and L. braziliensis protozoans) in the subject, by generating an immune response in the subject directed to at least one carbohydrate, such as the α-Gal epitope. In one embodiment, the methods of the invention induce production of α-Gal epitope-specific antibodies in the subject. In another embodiment, the methods of the invention induce immunity against Leishmania protozoans (e.g., L. mexicana, L. major, L. tropica, L. amazonensis, L. infantum, L. donovani and L. braziliensis protozoans) in the subject, by generating an immune response in the subject directed to at least one peptide that is associated with the Leishmania protozoan, including on the surface of the parasite.

In one embodiment, the methods of the invention prevent a Leishmania protozoan (e.g., L. mexicana, L. major, L. tropica, L. amazonensis, L. infantum, L. donovani and L. braziliensis protozoan) related disease or disorder in a subject in need thereof. In one embodiment, the methods of the invention comprise administering to the subject a composition comprising at least a portion of at least one carbohydrate or peptide associated with a Leishmania protozoan (e.g., L. mexicana, L. major, L. tropica, L. amazonensis, L. infantum, L. donovani and L. braziliensis protozoans) to a subject. In another embodiment, the methods of the invention comprise administering to the subject a composition comprising a nucleic acid sequence encoding at least one peptide associated with Leishmania protozoan (e.g., L. mexicana, L. major, L. tropica, L. amazonensis, L. infantum, L. donovani and L. braziliensis protozoan) to a subject.

The present invention provides compositions, including carbohydrates, peptides, peptide-based molecules, antibodies, nucleotides, vectors, and vaccines, that when administered to a subject, elicit an immune response directed against a Leishmania protozoan (e.g., L. mexicana, L. major, L. tropica, L. amazonensis, L. infantum, L. donovani and L. braziliensis protozoans), including an immune response directed against at least one carbohydrate, peptide or peptide-based molecule associated with a Leishmaniasis protozoan (e.g., L. mexicana, L. major, L. tropica, L. amazonensis, L. infantum, L. donovani and L. braziliensis protozoans). Further, when the compositions are administered to a subject, they may elicit an immune response that serves to protect the subject against diseases or disorders associated with a leishmaniasis (due to e.g., L. mexicana, L. major, L. tropica, L. amazonensis, L. infantum, L. donovani and L. braziliensis protozoans) infection, including but not limited to cutaneous, visceral, and mucocutaneous leishmaniasis.

In one embodiment, the present invention provides compositions that are useful as immunomodulatory agents, for example, in initiating or stimulating an immune response and in preventing or diminishing leishmaniasis-related (e.g., L. mexicana, L. major, L. tropica, L. amazonensis, L. infantum, L. donovani and L. braziliensis-related) disease or disorder, including but not limited to cutaneous, visceral, and mucocutaneous leishmaniasis. In various embodiments, the immunomodulatory agents comprise at least one carbohydrate, peptide or peptide-based molecule associated with L. amazonensis or L. infantum. In various embodiments, the immunomodulatory agents comprise an α-Gal epitope, and fragments or variants thereof. In various embodiments, the immunomodulatory agents comprise antibodies, such as for example and not limitation, antibodies against carbohydrates, peptides and/or peptide-based molecules, such as those found on the surface of Leishmania parasites, e.g., the α-Gal epitope.

Carbohydrates, peptides and peptide-based molecules associated with Leishmania protozoans can be used as immunostimulatory agents to induce the production of antibodies to protect, prevent, or reduce the likelihood of developing a leishmaniasis (due to e.g., L. mexicana, L. major, L. tropica, L. amazonensis, L. infantum, L. donovani and L. braziliensis protozoans) induced disease or disorder. In one embodiment, the composition of the invention comprises an α-Gal epitope, and fragments or variants thereof. At least one carbohydrate and/or peptide-based molecule associated with L. amazonensis or L. infantum, useful in eliciting an immune response, can each be used alone or in any combination for eliciting an immune response.

The present invention also provides polynucleotides that encode the peptides and peptide-based molecules described herein. Therefore, in one embodiment, the composition of the invention comprises a nucleic acid sequence encoding the peptides as described herein, or a variant thereof. At least one peptide associated with Leishmania protozoans, useful in eliciting an immune response, can each be used alone or in any combination for eliciting an immune response.

In one embodiment, the carbohydrates, peptides or peptide-based molecules or combinations thereof of the present invention are capable of generating a specific immune response. In another embodiment, the carbohydrates peptides or peptide-based molecules or combinations thereof of the present invention are capable of generating specific antibodies.

The carbohydrates, peptides and peptide-based molecules of the invention can be prepared as a combination, which includes two or more of the carbohydrates or peptides or peptide-based molecules of the invention, for use as a vaccine for the reduction, prevention, or treatment of leishmaniasis (caused by e.g., L. mexicana, L. major, L. tropica, L. amazonensis, L. infantum, L. donovani and L. braziliensis) infection, including but not limited to cutaneous, visceral, and mucocutaneous leishmaniasis. The carbohydrates. peptides and peptide-based molecules may be in a cocktail or may be conjugated to each other using standard techniques. For example, the carbohydrates, peptides and/or peptide-based molecules of the invention may be bound to an immunogenic protein nanoparticle, such as a virus-like particle (VLP). The peptides in the combination may be the same or different.

In another embodiment, antibodies to the carbohydrates, peptides or peptide-based molecules of the invention may be administered in order to generate an immune response (i.e., passive immunization). Therefore, compositions of the invention also comprise such antibodies, as well as adjuvants, carriers, and other pharmaceutically acceptable agents. Non-limiting examples of antibodies useful against Leishmania protozoans are described in, e.g., U.S. Pat. Nos. 4,992,273, 8,425,919, 6,375,955, WO1983/001785, U.S. Pat. Nos. 8,669,100, 9,909,114, and 8,986,711.

### Immunogenic Protein Nanoparticles

In some embodiments, the carbohydrates and peptides of the invention can be bound to an immunogenic protein nanoparticle (as described in, e.g., WO2004/084939, U.S. Pat. Nos. 7,959,928, 5,871,747, WO2018/031771, U.S. Pat. No. 9,522,180, US2004/0014708, US2005/0118275, Kruger, D. H.; Ulrich, R.; Gerlich, W. H., Chimeric virus-like particles as vaccines. Biol. Chem. 1999, 380, 275-276; Chackerian, B., Virus-like particles: flexible platforms for vaccine development. Exp. Rev. Vaccines 2007, 6, 381-390; Jennings, G. T.; Bachmann, M. F., Coming of age of virus-like particle vaccines. Biol. Chem. 2008, 389, 521-536; Bachmann, M. F.; Jennings, G. T., Vaccine delivery: a matter of size, geometry, kinetics and molecular patterns. Nat. Rcv. Immunol. 2010, 10, 787-796; Mohsen, M. O.; Gomes, A. C.; Vogel, M.; Bachmann, M. F., Interaction of Viral Capsid-Derived Virus-Like Particles (VLPs) with the Innate Immune System. Vaccines 2018, 6, 37; Strable, E.; Finn, M. G., Chemical Modification of Viruses and Virus-Like Particles. Curr. Top. Microbiol. Immun. 2009, 327, 1-22; Kaltgrad, E.; Sen Gupta, S.; Punna, S.; Huang, C.-Y.; Chang, A.; Wong, C.-H.; Finn, M. G.; Blixt, O., Anti-Carbohydrate Antibodies Elicited by Polyvalent Display on a Viral Scaffold. ChemBioChem 2007, 8, 1455-1462; Astronomo, R. D.; Kaltgrad, E.; Udit, A. K.; Wang, S.-K.; Doores, K. J.; Huang, C.-Y.; Pantophlet, R.; Paulson, J. C.; Wong, C.-H.; Finn, M. G.; Burton, D. R., Defining Criteria for Oligomannose Immunogens for HIV Using Icosahedral Virus Capsid Scaffolds. Chem. Biol. 2010, 17, 357-370; Yin, Z.; Comellas-Aragones, M.; Chowdhury, S.; Bentley, P.; Kaczanowska, K.; BenMohamed, L.; Gildersleeve, J.; Finn, M. G.; Huang, X., Boosting Immunity to Small Tumor-Associated Carbohydrates with Bacteriophage Qβ Capsids. ACS Chem. Biol. 2013, 8, 1253-1262; Brito, C. R. N.; McKay, C. S.; Azevedo, M. A.; Santos, L. C. B.; Nunes, D. F.; D'Avila, D. A.; Rodrigues de Cunha, G. M.; Almeida, I. C.; Gazzinelli, R. T.; Galvao, L. M. C.; Chiari, E.; Sanhueza, C. A.; Finn, M. G.; Marques, A. F., Virus-like particle display of the α-Gal epitope for diagnostic assessment of Chagas disease. ACS Infect. Dis. 2016, 2, 917-922; Yin, Z.; Dulaney, S.; McKay, C. S.; Baniel, C.; Kaczanowska, K.; Ramadan, S.; Finn, M. G.; Huang, X. F., Chemical Synthesis of GM2 Glycans, Bioconjugation with Bacteriophage Qb, and the Induction of Anticancer Antibodies. ChemBioChem 2016, 17, 174-180). In some embodiments, the immunogenic protein nanoparticle is a virus-like particle (VLP) (as described in, e.g., US2005/0009008, U.S. Pat. Nos. 6,077,662, 5,420,026, 7,517,520). Non-limiting examples of virus-like particles include bacteriophages, such as leviphages. Non-limiting examples of leviphages include Qβ and PP7 VLPs. The immunogenic nanoparticles may range from 10-100 nm in diameter, preferably 20-50 nm in diameter.

The carbohydrates and peptides of the invention can be bound to the immunogenic protein nanoparticle by one or more covalent or non-covalent bonds. In some embodiments, the carbohydrates and peptides of the invention can be bound or fused to the surface of the immunogenic protein nanoparticle. Preferably, the bonds are covalent bonds. In some embodiments, the carbohydrates and peptides of the invention are bound to the immunogenic protein nanoparticle by a peptide linker. Non-limiting examples of peptide linkers include molecules with chain lengths from 1 to 100 atoms, which can comprise alkyl chains, aryl groups, peptides, oligo(ethylene glycol) chains, peptoids, oligo- or polyesters, oligo- or polyacrylates, oligo- or polyamides.

In some embodiments, the immunogenic protein nanoparticle comprises 180 copies of the same protein that spontaneously self-assemble.

In some embodiments, the immunogenic protein nanoparticle is assembled in a bacterial cell, such as E. coli. The immunogenic protein nanoparticle can comprise about 2 kb of RNA inside the immunogenic protein nanoparticle, some of which is RNA generated from the expression vector for producing the immunogenic protein nanoparticle. Without wishing to be bound by theory, it is suggested that the RNA comprised or packaged in the immunogenic protein nanoparticle contributes to the immune response against the immunogenic protein nanoparticle.

### Vaccine Compositions

Provided is a vaccine composition comprising at least one carbohydrate, peptide and/or peptide-based molecule associated with a Leishmania protozoan (e.g., L. mexicana, L. major, L. tropica, L. amazonensis, L. infantum, L. donovani and L. braziliensis protozoan). Exemplary carbohydrates may comprise the α-Gal epitope (e.g., the Gal-α1,3-Gal(3 disaccharide and the Gal-α1,3-Gal-β1,4-GlcNAc trisaccharide). Exemplary peptides and/or peptide-based molecules may comprise any of (1) glycopeptides; (2) peptides of 6 to 50 amino acids in length, of sequences selected from proteins expressed by the Leishmania protozoan, including peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length; (3) protein domains (generally larger than 50 amino acids) and/or whole proteins expressed by the Leishmania protozoan, and combinations thereof; and/or (4) peptides of length 6-50 amino acids in length that are not found in proteins produced by Leishmania protozoans, but that have three-dimensional structures which mimic peptide or glycan structures produced by the protozoan, including peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length. More than one such peptide and/or peptide-based molecule may be used in compositions of the invention. Combinations of any or all of these four classes may also be included in compositions of the invention.

The vaccine composition may further comprise additional antigens associated with Leishmaniasis protozoans, such as antigens associated with the promastigote, amastigote forms. The vaccine composition may be useful in treating or preventing leishmaniasis infections, including cutaneous, visceral, and mucocutaneous leishmaniasis.

The carbohydrate may be the α-Gal epitope (e.g., the Gal-α1,3-Galβ disaccharide and the Gal-α1,3-Gal-β1,4-GlcNAc trisaccharide) and fragments or variants thereof.

In various embodiments, the vaccine composition is effective to induce an immune response to the antigen in a cell, tissue or subject (e.g., a human). In some embodiments, the vaccine induces a protective immune response in the subject. As used herein, an “immunological composition” may comprise, by way of examples, an antigen (e.g., a polypeptide), a nucleic acid encoding an antigen (e.g., an antigen expression vector), or a cell expressing or presenting an antigen. In some embodiments, the vaccine composition comprises or encodes all or part of any carbohydrate or peptide antigen described herein, or an immunologically functional equivalent thereof. In other embodiments, the vaccine composition is in a mixture that comprises an additional immunostimulatory agent or nucleic acids encoding such an agent. Immunostimulatory agents include but are not limited to an additional antigen, an immunomodulator, an antigen presenting cell or an adjuvant. Non-limiting examples of suitable adjuvants include cholera toxin, salmonella toxin, alum and such, but are not limited thereto. In other embodiments, one or more of the additional agent(s) is covalently bonded to the immunogenic protein nanoparticle or to the antigen or an immunostimulatory agent, in any combination. In certain embodiments, the vaccine composition is conjugated to or comprises HLA anchor motif amino acids.

In various embodiments, the vaccine composition comprises at least one carbohydrate, peptide and/or peptide-based molecule associated with a Leishmania protozoan that can be administered to a subject to induce an immune response. In various embodiments, the vaccine of the invention comprises a carbohydrate selected from the group consisting of the Gal-α1,3-Galβ disaccharide and/or the Gal-α1,3-Gal-β1,4-GlcNAc trisaccharide, and fragments or variants thereof. In various embodiments, the vaccine composition comprises at least one antibody against at least one carbohydrate, peptide and/or peptide-based molecule associated with a Leishmania protozoan that can be administered to a subject to induce an immune response.

In one embodiment, the vaccine composition is administered in combination with an adjuvant. In another embodiment, the vaccine is administered in the absence of an adjuvant.

In a non-limiting example, a nucleic encoding an antigen might also be formulated with an adjuvant. The various compositions described herein may further comprise additional components. For example, one or more vaccine components may be comprised in a lipid or liposome.

In another non-limiting example, a vaccine may comprise one or more adjuvants. Non-limiting examples of suitable adjuvants include cholera toxin, salmonella toxin, alum and such, but are not limited thereto. A vaccine of the present invention, and its various components, may be prepared and/or administered by any method disclosed herein or as would be known to one of ordinary skill in the art, in light of the present disclosure.

In one embodiment, the peptide or peptide-based vaccine of the invention includes, but is not limited to, at least one peptide or peptide-based, or a fragment thereof, optionally mixed with adjuvant substances. In some embodiments, the peptide or peptide-based is introduced together with an antigen presenting cell (APC). The most common cells used for the latter type of vaccine are bone marrow and peripheral blood derived dendritic cells, as these cells express costimulatory molecules that help activation of T cells. WO 00/06723 discloses a cellular vaccine composition which includes an APC presenting tumor associated antigen polypeptides. Presenting the polypeptide can be effected by loading the APC with a polynucleotide (e.g., DNA, RNA) encoding the polypeptide or loading the APC with the polypeptide itself.

Thus, the present invention also encompasses a method of inducing immunity against Leishmania protozoans (e.g., L. mexicana, L. major, L. tropica, L. amazonensis, L. infantum, L. donovani and L. braziliensis protozoans) using one or more of the carbohydrates, peptides and/or peptide-based molecules described herein. When a certain carbohydrates, peptides and/or peptide-based molecules or combination of carbohydrates, peptides and/or peptide-based molecules induces an immune response to Leishmania protozoans (e.g., L. mexicana, L. major, L. tropica, L. amazonensis, L. infantum, L. donovani and L. braziliensis protozoans) after inoculation into an animal, the carbohydrates, peptides and/or peptide-based molecules or combination of carbohydrates, peptides and/or peptide-based molecules is understood to have an immunity inducing effect. The induction of immunity to Leishmania protozoans (e.g., L. mexicana, L. major, L. tropica, L. amazonensis, L. infantum, L. donovani and L. braziliensis protozoans) by carbohydrates, peptides and/or peptide-based molecules or combination of carbohydrates, peptides and/or peptide-based molecules can be detected by observing the response of the immune system, in vivo or in vitro, by the host against the carbohydrate, peptide or combination of carbohydrates and peptides.

In another embodiment, the methods of the invention comprise administering to the subject a bacterium or virus comprising a carbohydrate and/or a nucleic acid sequence encoding at least one peptide or peptide-based molecule associated with a Leishmania protozoan. In another embodiment, the methods of the invention comprise administering to the subject a bacterium or virus expressing a carbohydrate and/or at least a portion of at least one peptide or peptide-based molecule associated with a Leishmania protozoan. In another embodiment, the methods of the invention comprise administering to the subject a bacterium or virus comprising a carbohydrate and/or at least a portion of at least one peptide or peptide-based molecule associated with a Leishmania protozoan.

In another embodiment, the methods of the invention comprise administering to the subject an antibody against a carbohydrate and/or a peptide or peptide-based molecule associated with a Leishmania protozoan. In another embodiment, the methods of the invention comprise administering to the subject an immunogenic fragment of an antibody against a carbohydrate and/or a peptide or peptide-based molecule associated with a Leishmania protozoan.

The induction of immunity to Leishmania protozoans (e.g., L. mexicana, L. major, L. tropica, L. amazonensis, L. infantum, L. donovani and L. braziliensis protozoans) by carbohydrates, peptides and/or peptide-based molecules or combination of carbohydrates, peptides and/or peptide-based molecules can be further confirmed by observing the induction of antibody production against the protozoan. For example, when antibodies against a carbohydrate, peptide and/or peptide-based molecule or combination of carbohydrates, peptides and/or peptide-based molecules are induced in a laboratory animal immunized with the carbohydrates, peptides and/or peptide-based molecules or combination of carbohydrates, peptides and/or peptide-based molecules, and when a Leishmania protozoan-associated disease or disorder is suppressed by those antibodies, the carbohydrates, peptides and/or peptide-based molecules or combination of carbohydrates, peptides and/or peptide-based molecules are understood to induce anti-Leishmania (e.g., L. mexicana, L. major, L. tropica, L. amazonensis, L. infantum, L. donovani and L. braziliensis protozoans) immunity.

Leishmania (e.g., L. mexicana, L. major, L. tropica, L. amazonensis, L. infantum, L. donovani and L. braziliensis) immunity can be induced by administering a vaccine of the invention. Induction of Leishmania (e.g., L. mexicana, L. major, L. tropica, L. amazonensis, L. infantum, L. donovani and L. braziliensis) immunity may allow for treatment and prevention of pathologies associated with leishmaniasis, including cutaneous, visceral, and mucocutaneous leishmaniasis. Thus, the invention provides a method for treating or preventing infection by a Leishmania protozoan, such as but not limited to, leishmaniasis, including cutaneous, visceral, and mucocutaneous leishmaniasis. The therapeutic compounds or compositions of the invention may be administered prophylactically or therapeutically to subjects suffering from, at risk of developing, or susceptible to developing, leishmaniasis, including cutaneous, visceral, and mucocutaneous leishmaniasis. Such subjects may be identified using standard clinical methods. In the context of the present invention, prophylactic administration occurs prior to the manifestation of overt clinical symptoms of disease, such that a disease or disorder is prevented or alternatively delayed in its progression.

The carbohydrates, peptides and/or peptide-based molecules or combination of carbohydrates, peptides and/or peptide-based molecules of the invention having immunological activity, or a polynucleotide or vector encoding such a peptide, peptide-based molecule or combination thereof, may optionally be combined with an adjuvant. An adjuvant refers to a compound that enhances the immune response against the carbohydrates, peptides and/or peptide-based molecules or combination of carbohydrates, peptides and/or peptide-based molecules when administered together (or successively) with the carbohydrate, peptide and/or peptide-based molecule having immunological activity. Non-limiting examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

In various embodiments, the treatment of Leishmania protozoan (e.g., L. mexicana, L. major, L. tropica, L. amazonensis, L. infantum, L. donovani and L. braziliensis protozoan) infection in a subject is accomplished through passive antibody therapy (i.e., the transfer of antibodies to the of Leishmania protozoan (e.g., L. mexicana, L. major, L. tropica, L. amazonensis, L. infantum, L. donovani and L. braziliensis protozoan) infected subject). In various embodiments, the passive antibody therapy is used in combination with an antibiotic therapy, such as a therapy comprising use of an anti-fungal drug, an anti-viral drug, an anti-parasitic drug, an anti-protozoal drug, an anti-helminthic drug, or a combination thereof. When used in combination, the antibiotic therapy can be administered before, during or after the administration of the antibody compositions of the invention.

### Administration

In one embodiment, the methods of the present invention comprise administering to a subject a composition comprising at least one carbohydrate, peptide and/or peptide-based molecule or combination of carbohydrates, peptides and/or peptide-based molecules, and/or at least one polynucleotide encoding at least one peptide or peptide-based molecule of the invention, and/or at least one antibody or immunogenic fragment thereof against a carbohydrate, peptide and/or peptide-based molecule associated with a Leishmania protozoan. Administration of the composition can comprise, for example, intramuscular, intravenous, peritoneal, subcutaneous, intradermal, as well as topical administration.

The actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

In certain circumstances it may be desirable to deliver the compositions disclosed herein parenterally, intravenously, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In most cases the form must be sterile and must be fluid to the extent that easy syringability exists. It can be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Appropriate doses and methods of administration for these purposes can be readily determined by a skilled artisan using available knowledge in the art and/or routine techniques. Routes and frequency of administration, as well as dosage, for the above aspects of the present invention may vary from individual to individual and may parallel those currently being used in immunization against other infections, including protozoan, viral and bacterial infections. For example, in one embodiment, between 1 and 12 doses of compositions according to the invention are administered over a 1 year period. Booster vaccinations may be given periodically thereafter as needed or desired. Of course, alternate protocols may be appropriate for individual patients. In a particular embodiment, a suitable dose is an amount of a composition according to the invention that, when administered as described above, is capable of eliciting an immune response in an immunized patient sufficient to protect the patient from leishmaniasis caused by Leishmania species such as L. mexicana, L. major, L. tropica, L. amazonensis, L. infantum, L. donovani and L. braziliensis for at least 1-2 years. In general, the amount of a composition according to the invention present in a dose ranges from about 100 ng to about 1 mg per kg of host, typically from about 10 μg to about 100 μg. Suitable dose sizes will vary with the size of the patient, but will typically range from about 0.1 mL to about 5 mL.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Pharmaceutical Compositions

The present invention includes the treatment of Leishmania protozoan (e.g., L. mexicana, L. major, L. tropica, L. amazonensis, L. infantum, L. donovani and L. braziliensis protozoan) infection in a subject by the administration of a therapeutic composition of the invention to a subject in need thereof. In one embodiment, the infection includes cutaneous, visceral, and mucocutaneous leishmaniasis. In one embodiment, the therapeutic composition of the invention for the treatment of Leishmania protozoan (e.g., L. mexicana, L. major, L. tropica, L. amazonensis, L. infantum, L. donovani and L. braziliensis protozoan) infection is at least one antibody that specifically binds to at least one carbohydrate and/or peptide associated with Leishmania protozoan. In various embodiments, the therapeutic composition comprises at least one antibody that specifically binds to at least one of the group consisting of the Gal-α1,3-Galβ disaccharide and/or the Gal-α1,3-Gal-β1,4-GlcNAc trisaccharide, and fragments or variants thereof. In various embodiments, the therapeutic composition comprises at least one antibody that specifically binds to a peptide associated with a Leishmania protozoan. In various embodiments, the treatment of Leishmania protozoan infection in a subject is accomplished through passive antibody therapy (i.e., the transfer of antibodies to the Leishmania protozoan-infected subject).

Administration of the therapeutic composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

Vaccine compositions may further comprise a pharmaceutically acceptable carrier, diluent or excipient to form a pharmaceutical formulation, or unit dosage form. The total active ingredients (e.g., carbohydrates and peptides) in such formulations include from 0.1 to 99.9% by weight of the formulation. The active ingredients (e.g., carbohydrates and peptides) for administration may be present as a powder or as granules; as a solution, a suspension or an emulsion.

Pharmaceutical formulations containing the compositions of the invention can be prepared by procedures known in the art using well known and readily available ingredients. The compositions of the invention can also be formulated as solutions appropriate for parenteral administration, for instance by intramuscular, subcutaneous or intravenous routes.

Thus, the composition may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Identification of the α-Gal Epitope in Leishmania Species and Development of a Murine Vaccine

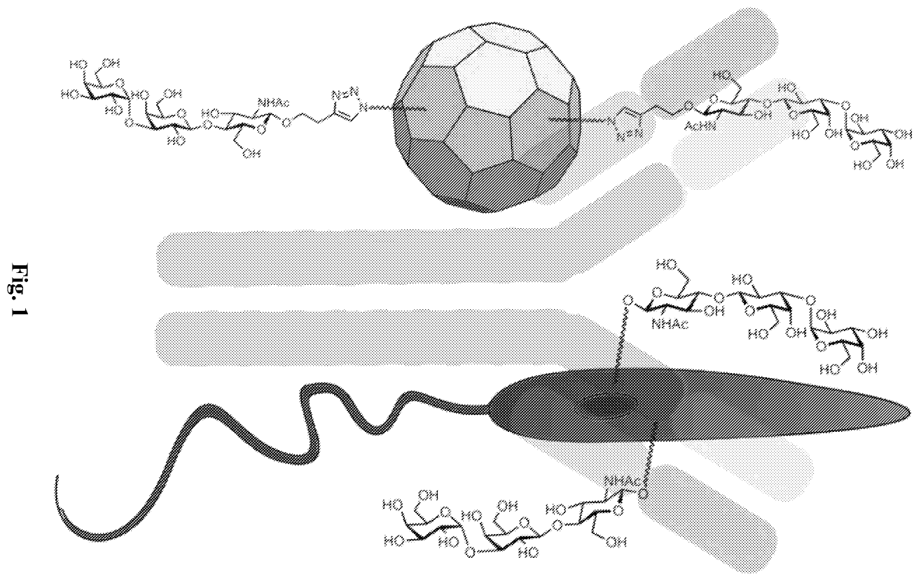

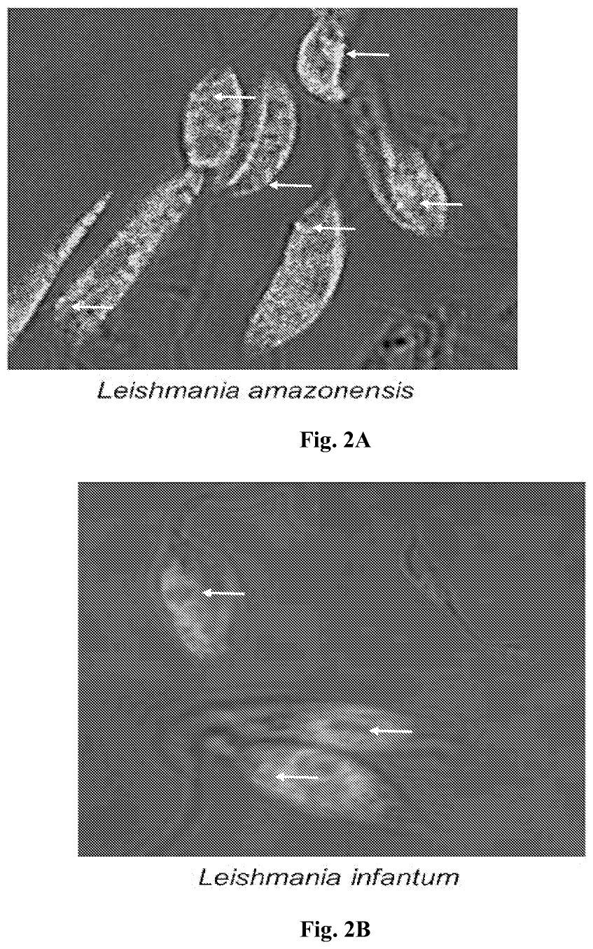

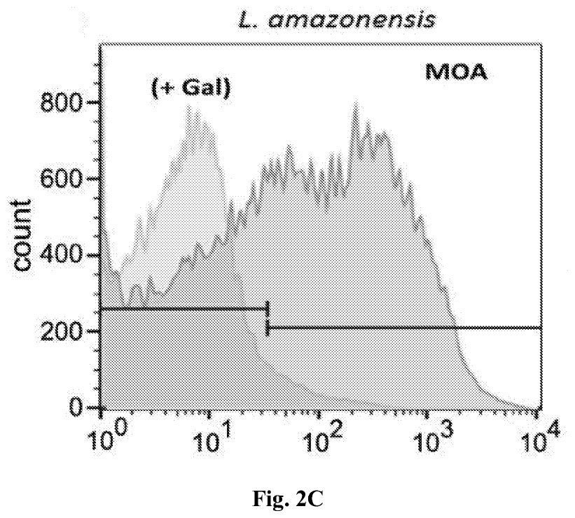

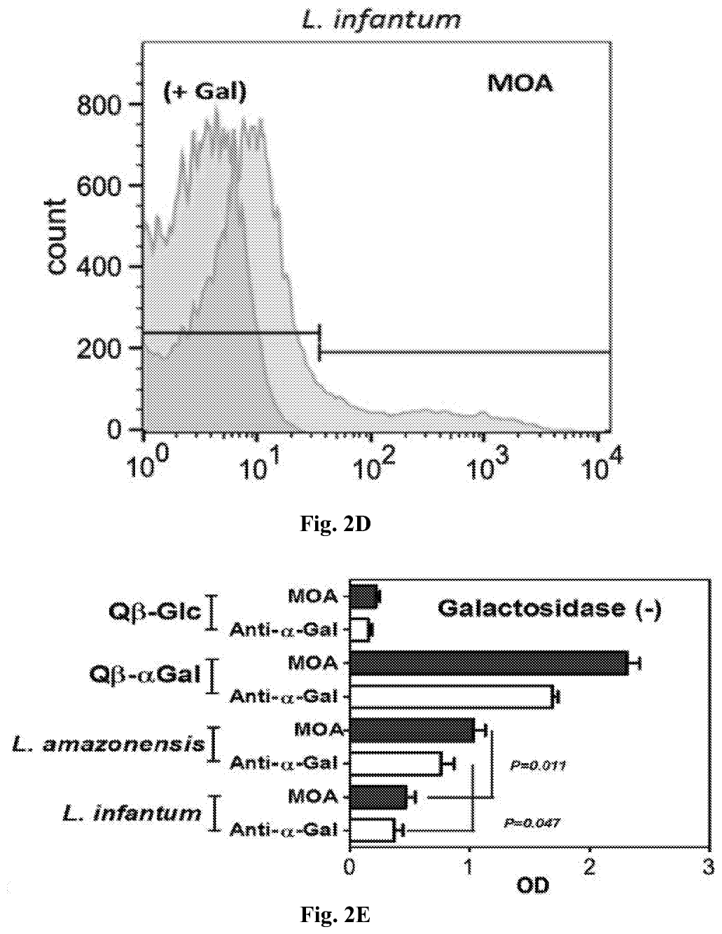

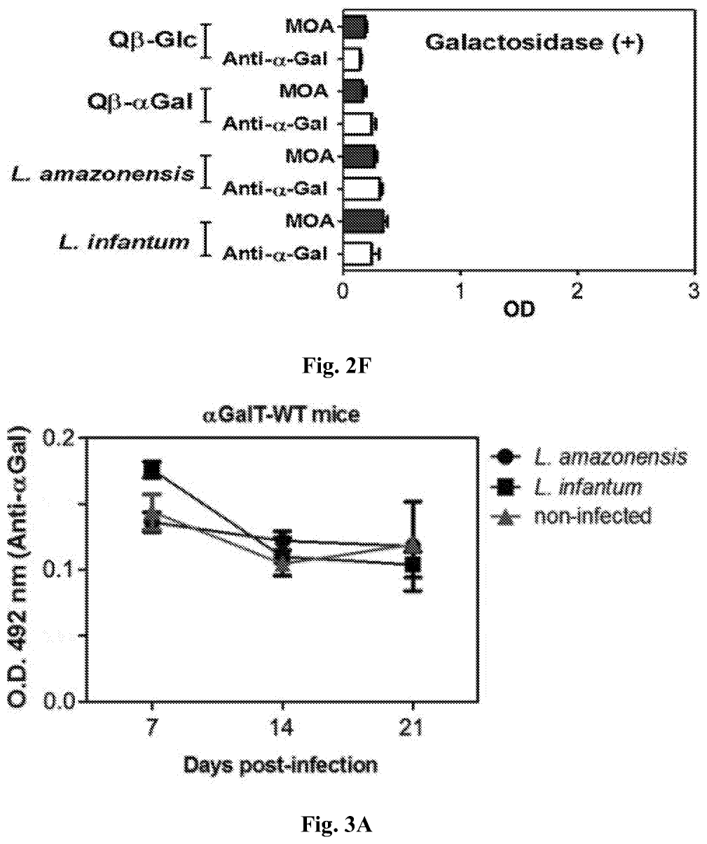

The presence of the α-Gal epitope was examined on the promastigote forms of L. infantum (MCAN/BR/2002/BH401) and L. amazonensis (IFLA/BR/1967/PH8) (FIG. 1). The Marasmius oreades agglutinin (MOA) lectin, which binds strongly to the α-Gal trisaccharide, the related type B branched trisaccharide, and the Gal-α1,3-Gal disaccharide (but not the isomeric α1,2-α1,4- and α1,6-disaccharides (23,24) was used as the key reagent for these studies. Promastigote cells (10⁸ per mL) of L. infantum and L. amazonensis were labeled with 3-5 μg/mL of fluorescein-labeled MOA and examined by fluorescence microscopy (25) and flow cytometry (26). Strong staining of the L. amazonensis cell surface, compared to moderate staining of L. infantum, was observed (FIG. 2A, 2B), which was confirmed by flow cytometry (FIG. 2C, 2D). The addition of a large excess (0.2 M) of galactose largely abrogated binding, as expected. Lastly, L. amazonensis and L. infantum lysates were plated and incubated either with biotinylated MOA or purified anti-α-Gal antibody in an ELISA-type assay (FIG. 2E) (27). The presence of α-Gal was confirmed for both species, but to a greater degree for L. amazonensis. Preincubation with an α-galactosidase enzyme reduced binding by more than 90% (FIG. 2F), suggesting that the terminal galactose residue is important in binding. Taken together, these results provide strong evidence that both L. infantum and L. amazonensis express the α-Gal epitope on their surfaces, the latter in greater amount or accessibility.

### Development of a Murine Model

In visceral leishmaniasis, caused by L. infantum, parasites are able to escape from the host immune system and reach internal organs such as liver or spleen, whereas in cutaneous leishmaniasis L. amazonensis usually stay at the site of the infection. C57BL/6 mice have been used to study pathogenesis of experimental cutaneous and visceral leishmaniasis (28). However, in spite of intense effort with this model, greater consistency is needed, particularly over multiple Leishmania strains and numbers of parasites used for infection. The presence of α-Gal on the parasite makes these experiments even less reliable as an indicator of the human response since α-Gal is a self-sugar in these wild-type C57BL/6 mice. Therefore, a new murine model of cutaneous and visceral leishmaniasis was needed.

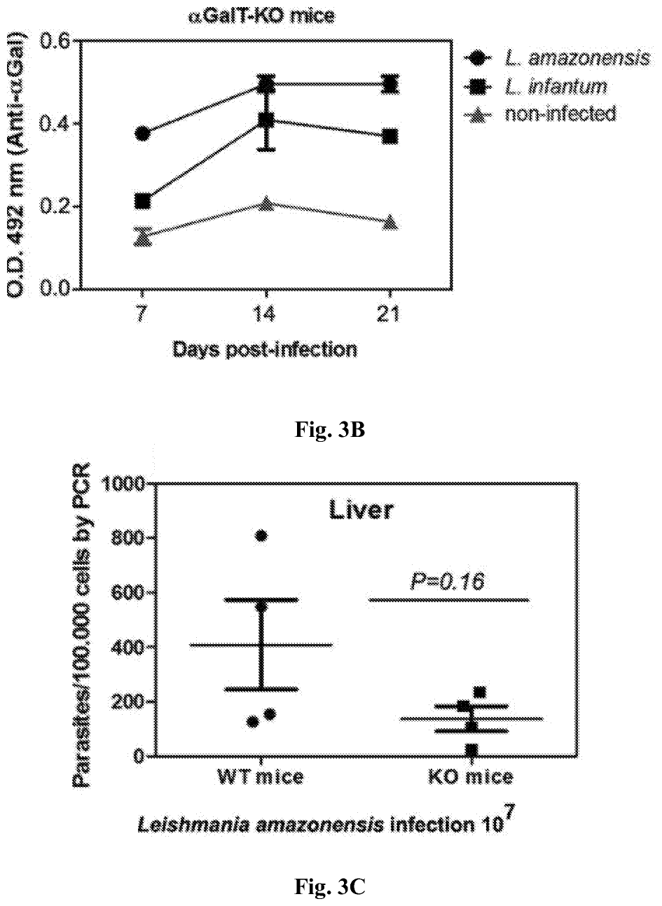

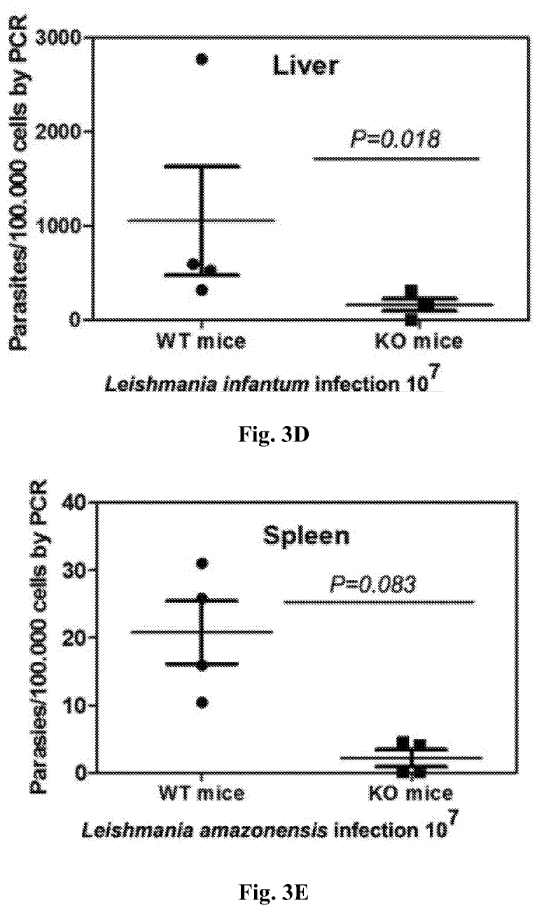

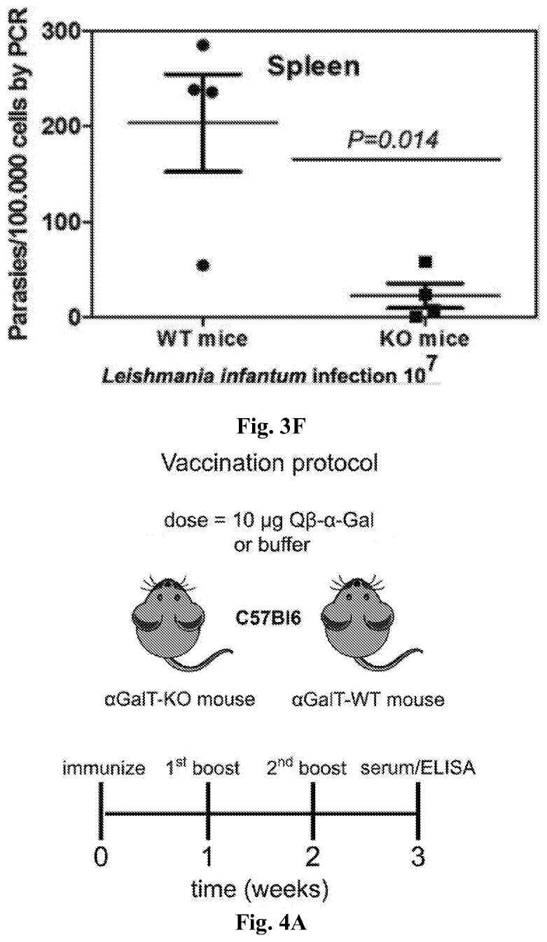

The response of α-GalT-KO versus α-GalT-WT mice to intraperitoneal infection with 10⁷ promastigotes of L. infantum and subcutaneously with the same number of L. amazonensis was compared. Blood samples for ELISA were collected at days 7, 14, and 21; liver and spleen samples were collected for quantitative PCR (qPCR) at 10 weeks post-infection. The Leishmania-infected α-GalT-WT produced levels of anti-α-Gal antibodies identical to uninfected controls, representing a negligible response against the α-Gal epitope (FIG. 3A). In contrast, α-GalT-KO mice infected with Leishmania produced anti-α-Gal antibodies to a moderate extent, and significantly greater than noninfected controls (FIG. 3B). Infection with L. amazonensis induced a more intense anti-α-Gal response, consistent with its higher level of α-Gal display. Parasite loads in liver and spleen were assessed by qPCR, with significantly greater variability for α-GalT-WT mice than α-GalT-KO mice. Differences in liver infection at 10 weeks were not significant for either Leishmania strain between the two mouse models (p>0.05, FIG. 3C, 3E), but both parasites were found in the spleens of α-GalT-WT mice to a significantly greater extent than α-GalT-KO mice (FIG. 3D, 3F).

These findings suggested that α-GalT-KO mice were more resistant than α-GalT-WT toward Leishmania infection, perhaps because of the anti-α-Gal antibodies produced by the knockout animals. The highly variable nature of the response of WT mice (FIGS. 3C, 3D, 3E, 3F) was expected given that many factors that contribute to the extent of infection in the absence of a controlling factor, including parasite dissemination from the site of injection and the draining lymph nodes to other organs (29-32).

### Development of a Murine Vaccine

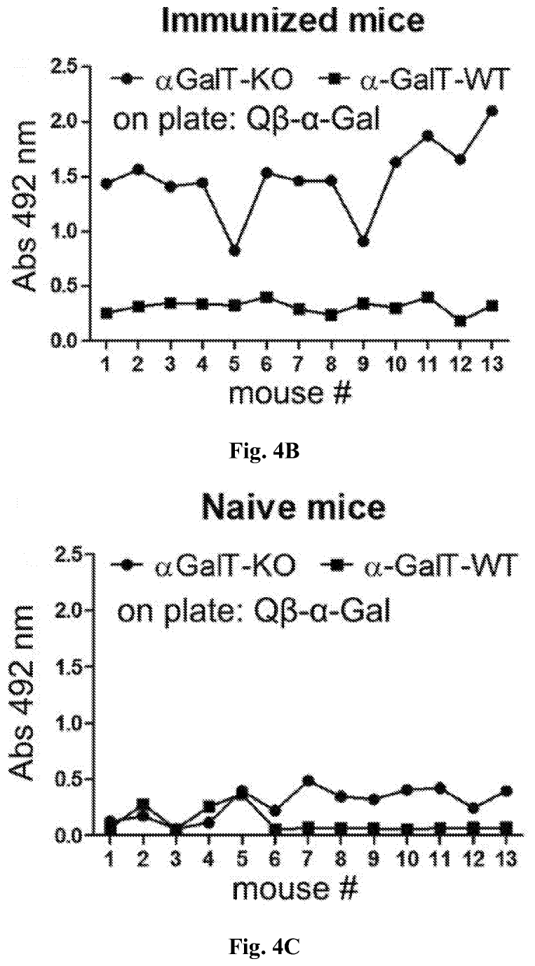

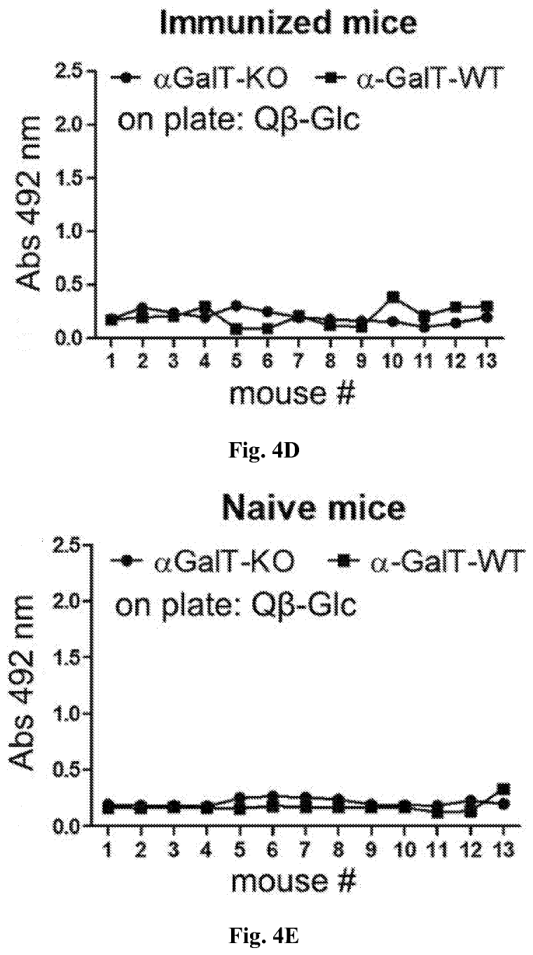

To further test the proposed role of an induced anti-α-Gal immune response, both mouse strains were immunized with the previously reported (33,34) Qβ virus-like particle bearing approximately 540 copies of the α-Gal trisaccharide, designated Qβ-α-Gal. It was suggested that high anti-α-Gal antibody levels would be generated against the glycoconjugate, and that this response would protect these mice more effectively from Leishmania infection. The immunization protocol is shown in FIG. 4A, in which groups of mice were immunized with 10 μg Qβ-α-Gal particles (containing approximately 1 μg of attached α-Gal) and boosted twice (at one-week intervals) with the same dose.

The immune response of these animals compared to nonimmunized control mice was assessed by ELISA against two reagents: the Qβ-α-Gal immunogen and an analogous particle (Qβ-Glc) bearing glucose moieties (approximately 500 per particle) in place of α-Gal. As shown in FIG. 4B, the immunized α-GalT-KO mice produced a strong IgG anti-α-Gal response, whereas α-GalT-WT gave no such response above background. The immune response against the carrier Qβ protein was not independently assessed, but is unlikely to have contributed significantly to the results, as evidenced by the modest intensities of ELISA signals against Qβ-Glc (FIG. 4D). As expected, nonimmunized mice showed no IgG antibodies against Qβ-α-Gal or Qβ-Glc particles (FIG. 4C, 4E).

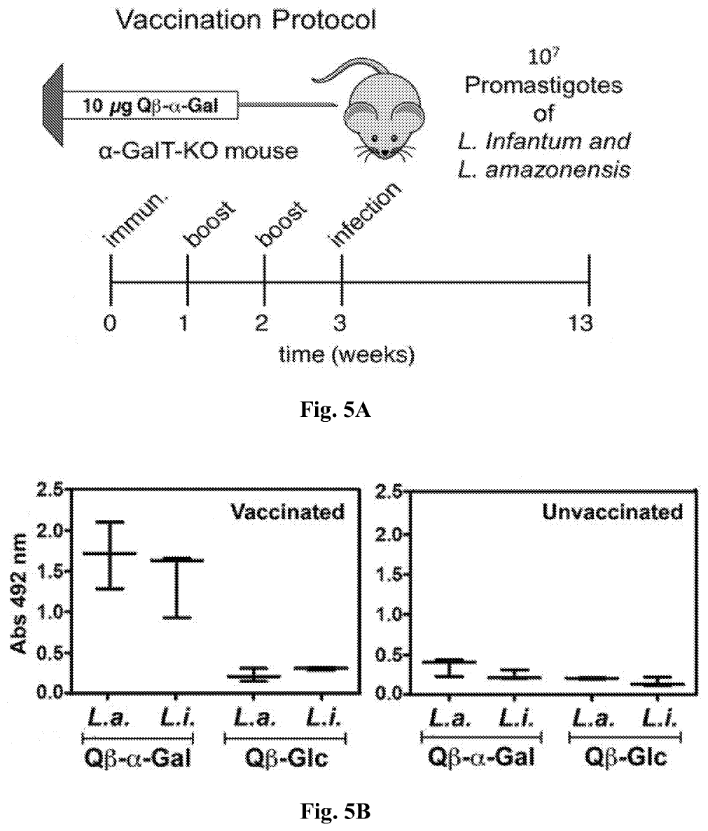

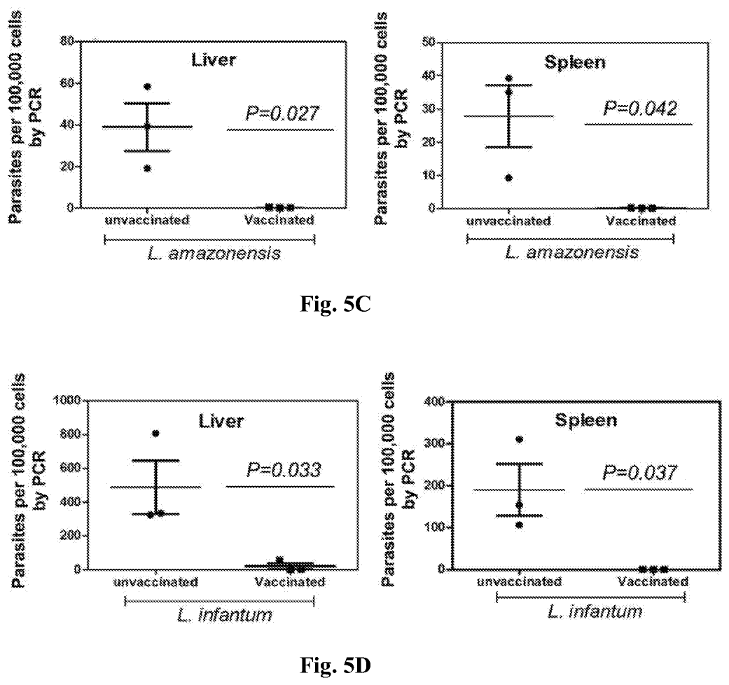

Following vaccination as above with Qβ-α-Gal (immunization+two boosts), groups of 3-5 mice (FIG. 5A) were assessed for anti-α-Gal antibody production (FIG. 5B) and then were infected with 10⁷ L. amazonensis or L. infantum by the appropriate route. Ten weeks later, the animals were sacrificed, and qPCR was performed on samples collected from liver and spleen from all groups. Parasite loads in both organs were found to be undetectable, or nearly so, for all vaccinated and infected α-GalT-KO mice, whereas the unvaccinated mice all harbored the parasite, in widely varying amounts (FIG. 5C-5D). Preliminary tests showed no difference between unvaccinated mice and mice immunized with Qβ-Glc in Leishmania challenge.

### DISCUSSION

Carbohydrates present on the surface of the Leishmania parasite have been proposed to accomplish several functions, many of which contribute to immune system evasion and pathogenesis (9,10). Since the α-Gal epitope has been found on many pathogens including organisms related to Leishmania, and since an α-Gal immune response had recently been found in Leishmania patients, the presence of α-Gal on two representative Leishmania species suggests that these carbohydrates could be used as vaccine candidates.

Antibodies against α-Gal have been reported to protect against experimental malaria infection (35), assist in the healing of burn wounds (36), improve immunogenicity in HIV vaccination (37), show lytic action against Trypanosoma cruzi parasites (38), and enhance immunogenicity in anticancer vaccine development (16). These diverse functions show that the anti-α-Gal immune response, while strong in all healthy humans, is also more complex than may be commonly appreciated. To study this for Leishmania, the α-galactosyltransferase knockout mouse developed herein can be a useful tool. Indeed, data presented herein showed that these animals are susceptible to immunization against causative organisms of cutaneous and visceral leishmaniasis with a conjugate vaccine that presents only the α-Gal carbohydrate in common with the parasites.

### Materials and Methods

### Virus-Like Particles

Qβ virus-like particles (of “wild type” sequence) were prepared, purified (39), chemically derivatized (34), and characterized as previously described.

### Mice

All animals and experiments were handled in strict accordance with the guidelines of the Research Ethics Committee of the UFMG, approved under the protocol number 137/2011. Female C57BL/6 mice (6-8 weeks old) having disrupted alleles of the α1,3-GalT gene 19 (α1,3GalT-KO) were used. These mice have the H-2b genetic background and are bred and maintained at the animal facility of Federal University of Minas Gerais, Belo Horizonte, Brazil.

### Fluorescence Microscopy and Flow Cytometry

Cells were fixed for 40 min in 2% paraformaldehyde in 0.1 mM phosphate buffer (pH 7.4), and washed three times in phosphate buffer at 4° C. for 15 min. Cells were then labeled with 3-5 μg/mL of MOA lectin-fluorescein isothiocyanate (FITC) for 60 min, washed, mounted on slides with Mowiol reagent (DABCO, Sigma a 2,3%), and observed using a fluorescent Nikon Eclipse Ti, (USA), with fluorescence illumination and a FITC filter-barrier system. Pictures were taken with Kodak Tri-X pan 400 ASA film. Lectins conjugated with FITC were obtained from EY laboratories, San Mateo, Calif.

Fluorescent signals were quantified using a Becton Dickinson FACScan (Becton Dickinson), excitation 488 nm, emission 530 nm (±15 nm), adjusted to a fixed channel using standard Brite Beads (Coulter) prior to determining fluorescence. Samples were briefly vortexed before introduction to sheath fluid. Data acquisition and manipulation were performed with CellQuest and FlowJo version X.0.7 (Tree Star Inc.). Lectin binding was blocked by the addition of 0.2 M galactose to verify specificity.

### Mice Immunization for Antibody Detection

The competence of α1,3-GalT-KO mice, previously immunized, was verified for producing antibodies (primarily IgG) against α-Gal epitopes. Immunization was performed in groups of 13 mice by subcutaneous injection of 10 mg doses of Qβ-α-Gal or unmodified particle at the base of the tail (not injection into the tail vein), followed by subcutaneous boost injections of the same dose at 1-week intervals in the flank. A control group of 13 mice was similarly treated with unmodified Qβ virus-like particle.

Only α-GalT-KO mice were used for vaccination and parasite challenge given the tolerance of α-GalT-WT mice to α-Gal epitopes. 40 Groups of 3-5 female α-GalT-KO mice were immunized (initial and two boost injections) as described above; control groups received PBS only. One week after the third injection, serum antibody levels were checked by ELISA. Each animal was then challenged with 10⁷ Leishmania parasites prepared as described in Supporting Information: L. amazonensis, MCAN/BR/2002/BH401, causing cutaneous leishmaniasis, so injected into the footpad in 10 μL volume and L. infantum, MCAN/BR/2002/BH401, causing visceral leishmaniasis, so injected i.p. in 200 μL volume.

### Leishmania Parasites

Promastigote forms of reference strains PH8 for Leishmania amazonensis (IFLA/BR/1967/PH8), and BH401 for Leishmania infantum (MCAN/BR/2002/BH401) were used. The parasites were initially isolated from hamsters, Mesocricetus auratus, that had been infected for two months. L. amazonensis promastigotes were isolated from injured lesion in the muzzle and L. infantum isolated from spleen fragments and grown in the NNN (Novy, McNeal and Nicolle) medium with Schneider's (SIGMA®), supplemented with 2% urine, 1% vitamin solution (BME Vitamins 100×-SIGMA®), 1% L-glutamine (200 mM), 10% bovine fetal serum, 100 U/mL penicillin and 100 μg/mL streptomycin (Gibco®). The cultures were maintained in BOD at a temperature of 23° C. f 1° C. After expansion, the promastigote cells were preserved in a solution containing Middle Schneider's medium (SIGMA®), 10% Glycerol, and 20% of BSA, and the strains were kept in liquid nitrogen at “Laboratorio Fisiologia de Insetos Hematófagos—LFIH” Parasitology Department, Federal University of Minas Gerais, until use. Parasite species were confirmed by PCR/RFLP as described previously (41).

### Mice Vaccination and Infection

Groups of 3-5 female αGalT-KO mice (42) were immunized with 10 μg Qβ-α-Gal once per week for three weeks; control groups were vaccinated with 10 μg of unmodified Qβ virus-like particles in PBS. All injections were subcutaneous, the first dose at the base of the tail (rump) and the following doses in the flank. One week later (week 4), the antibody levels were checked in serum by ELISA against Qβ-α-Gal (with minimal response against Qβ-Glu, showing that the protein response was blocked or not detected). All immunized mice were then subjected to infectious challenge. Parasites for infection of mice were prepared using parasites in stationary phase. Parasites from culture were washed 3× with PBS, centrifuged for 5 min to 3,000 g and then resuspended in 1× sterile PBS with final volume required to inoculate 10 μL of L. amazonensis, and 200 μL L. infantum per animal, each delivering a dose of 1×10⁷ promastigote cells. Uninfected inoculation-control mice were administered the same volumes of sterile saline in the same manner.

### Protein Extracts

Total protein extracts were prepared from 1×10⁸ of promastigote form of L. infantum and L. amazonensis parasites. The parasites were centrifuged at 2000 g for 10 min, and washed twice with phosphate buffered saline (PBS) and suspended in 3 mL of Lysis buffer [20 mM of Hepes, 10 mM of KCl, 1.5 mM of MgCl2, 250 mM of sucrose, 1 mM of DTT, 0.1 mM PMSF and 300 μl protease inhibitor cocktail (GE Healthcare, Piscataway, USA)], submitted to 5 cycles of freezing (liquid N₂) and thawing (42° C.). Then, the extracts were subjected to centrifugation at 8000 g for 20 min at 4° C. The supernatant was collected and stored at −70° C. for upcoming experiments (43). The protein samples were dosed by colorimetric method of bicinchoninic Acid (BCA), using the kit “BCA Protein Assay Reagent” (Thermo Scientific, Waltham, USA) according to the manufacturer's recommendations.

### α-Gal Antigen Linked to Qβ-Virus Like Particle and Conjugate Preparation

Qβ virus-like particles were prepared and purified as described previously (44); details of VLP production and purification are given elsewhere (45). All particles were characterized by size-exclusion chromatography, dynamic light scattering (Wyatt DynaPro), microfluidic gel electrophoresis (Agilent Bioanalyzer 2100, using Protein 80 chips), and electrospray ionization mass spectrometry on an accurate-mass time-of-flight instrument (Agilent G6230B); representative samples were further examined by transmission electron microscopy and multiangle light scattering (Malvern Viscotec). In all cases, standard properties of size and composition were observed, with the particles showing narrow size distributions and high protein purity (less than 5% protein impurities detected). Protein concentrations in solution were measured with the BCA method (Protein Reagent Kit, Pierce, USA), standardized with bovine serum albumin. For conjugate preparation, α-Gal trisaccharide (α-Gal-OH, Carbosynth US, LLC, San Diego, Calif.; this compound was also made in house as previously described (46) and glucose were converted to their respective alkyne derivatives by Lewis acid-mediated glycosylation of 3-butyn-2-ol. Each alkyne was attached to Qβ virus-like particles by a two-step procedure in which the protein nanoparticle was first acylated with an azide-terminated N-hydroxysuccinimide ester and then addressed by copper-catalyzed azide-alkyne cycloaddition (47).

For convenience, the synthetic procedures were:

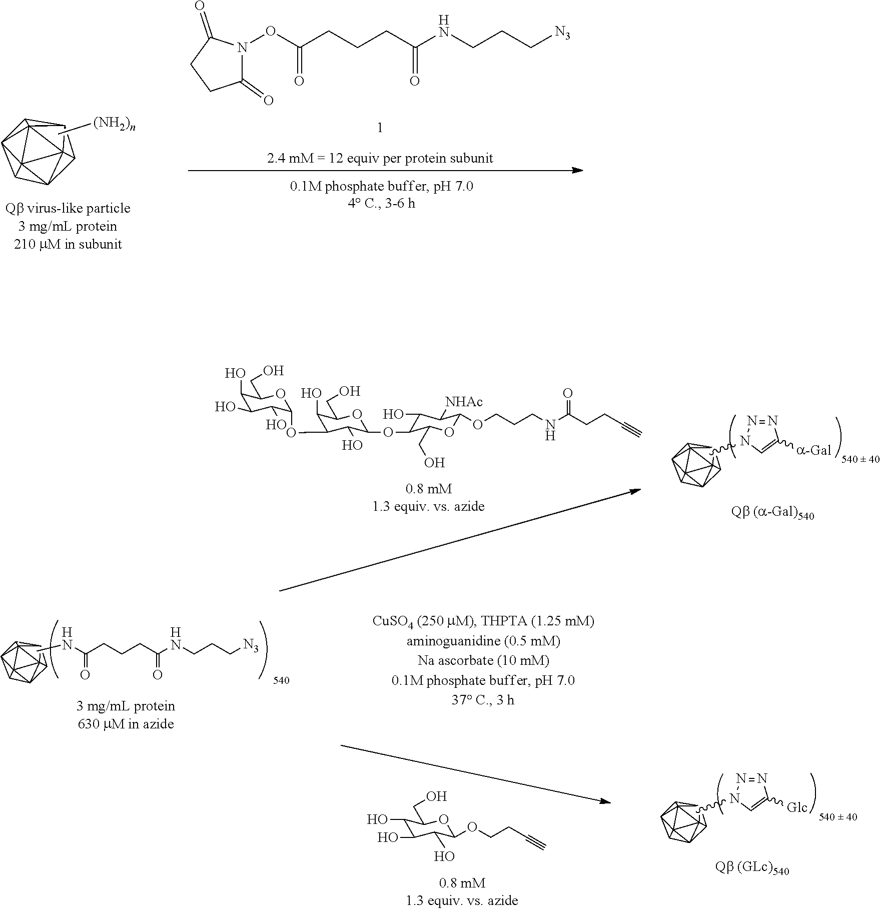

All particles are approximately 30 nm in diameter (dynamic light scattering); the product particles are very similar in physical properties (effective charge as determined by native gel electrophoresis; size as determined by dynamic light scattering and size-exclusion chromatography).

### ELISA for α-Gal Epitope Detection

High-binding ELISA plates (NUNC) were coated (overnight at 4° C., or 1 h at room temperature) with 10 μg/mL of L. infantum or L. amazonensis extracts (50 mM carbonate-bicarbonate buffer, pH 9.5). In place of the extracts, the Qβ-α-Gal antigen (2 μg/mL) was used as a positive α-Gal-displaying control; Qβ-Glc was used as negative control. After incubation and washing, free microplate binding sites were blocked with 2% bovine serum albumin (BSA, Sigma Aldrich) in phosphate buffered saline (PBS), pH 7.4. Each well was then incubated with one of two α-Gal binding reagents: (a) purified polyclonal mouse IgG anti-α-Gal antibody obtained as described elsewhere (43) (2 μg/mL), or (b) mushroom Marasmius oreades (MOA) lectin-HRP conjugate (EY Laboratories), which binds specifically to blood group B and terminal Gal-α1,3-Gal residues (5 μg/mL). After washing, each anti-α-Gal well (category a) was treated with 50 μL biotinylated anti-mouse IgG (1:2500 dilution in PBS with 2% BSA, Amersham, GE Healthcare Life Sciences, UK), washed, and then with 50 μL streptavidin-horseradish peroxidase (HRP) conjugate (1:4000 dilution, Amersham, UK) in PBS-BSA. All incubation steps were performed at 37° C. for 1 h. All wells were developed with 100 μL of peroxidase substrate SigmaFast™ OPD (o-phenylenediamine dihydrochloride and urea hydrogen peroxide, Sigma-Aldrich) with quenching of the reaction by addition of 2N sulfuric acid. Absorbance measurements were performed in a Multiskan GO instrument, using Skanit 3.2 software (Thermo Scientific). To determine antibody specificity, the VLP conjugates were placed on 96 wells plate and treated overnight at 28° C. with 0.1 U/well of α-galactosidase enzyme (from green coffee beans, Sigma-Aldrich G8507). After the incubation, the ELISA was performed as described.

### ELISA for Anti-α-Gal Antibody Detection

Serum samples from immunized mice were evaluated using the same ELISA procedure as above, coating the plates with Qβ-α-Gal (5 μg/mL). After incubation with serum samples (1 h at 37° C.) and washing, monoclonal goat anti-mouse IgG (Amersham) HRP conjugate was used as secondary antibody (1:4000 dilution), followed by peroxidase substrate and detection as above. To further validate that the observed response is specific to the α-Gal sugar, the Qβ-α-Gal reagent was plated and then treated overnight at 28° C. with 0.1 U/well of green coffee bean α-galactosidase (Sigma G8507). After the incubation, ELISA analysis gave little to no signal above background.

### Source of Samples for DNA Extraction

Mouse tissue were collected 10 weeks post-infection from spleen and liver of C57BL6 KO or WT mice infected by L. infantum (MCAN/BR/2002/BH401) and L. amazonensis (IFLA/BR/1967/PH8). The infection was performed as described above and naive mice were used as controls. Tissues were removed by using different scissors or scalpels to avoid cross-contamination and were minced with Potter grinders and then carefully homogenized in 1.5-ml microtubes with single-use blue pellet pestles (Polylabo, Paris, France) in phosphate-buffered saline.

Aliquots of the homogenates were stored at −80° C. until DNA extraction. DNA extraction was performed using the “Genomic DNA from tissue” kit (Macherey-Nagel, Duren, Germany) according to the manufacturer's recommendations. DNA was quantified using a NanoDrop 2000 spectrophotometer (Thermo Scientific).

### Quantitative PCR

Extraction of DNA and evaluation of spleen parasite load by qPCR. DNA was extracted from liver and spleen samples using NucleoSpin® Tissue (Macherey-Nagel) according to the manufacturer's instructions. The parasite load was calculated by qPCR according to a method described elsewhere with minor modifications (48,49).

The parasite burdens were estimated using primers amplifying a 90 bp fragment of a single-copy-number Leishmania DNA polymerase gene (GenBank: AF009147). The host's housekeeping β-actin gene was used as endogenous control in order to normalize initial DNA concentrations and to verify sample integrity. The primers were used to amplify a 307-bp fragment of β-actin. PCR was carried out in a final volume of 10 μL containing 2 pmol of each DNA polymerase primers, SYBR® Green (Applied Biosystems), 4 μL of DNA with a concentration of 5 ng/μL and enough volume of ultrapure water. Reactions were processed and analyzed in an ABI Prism 7500 Sequence Detection System (Applied Biosystems). The following steps were programmed: 95° C. for 10 min followed by 40 cycles at 95° C. for 15 s and 60° C. for 1 min. Parasite quantification for each spleen sample was calculated by interpolation from the standard curve included in the same run, performed in duplicate, and expressed as the number of parasites per 100,000 of host cells.

### REFERENCES

1. Bates P. A. Transmission of Leishmania metacyclic promastigotes by phlebotomine sand flies. Int. J. Parasitol. 2007, 37, 1097-1106.
2. Desjeux P. Leishmaniasis: current situation and new perspectives. Comp. Immunol. Microbiol. Infect. Dis. 2004, 27, 305-318.
3. Goihman-Yahr M. American mucocutaneous leishmaniasis. Dermatol. Clin. 1994, 12, 703-712.
4. Handman E. Leishmaniasis: current status of vaccine development. Clin. Microbiol. Rev. 2001, 14, 229-243.
5. Lukes J.; Mauricio I. L.; Schonian G.; Dujardin J. C.; Soteriadou K.; Dedet J. P.; Kuhls K.; Tintaya K. W.; Jirkil M.; Chocholova E.; Haralambous C.; Pratlong F.; Obornik M.; Horak A.; Ayala F. J.; Miles M. A. Evolutionary and geographical history of the Leishmania donovani complex with a revision of current taxonomy. Proc. Natl. Acad. Sci. U.S.A 2007, 104, 9375-9380.
6. Murray H. W.; Berman J. D.; Davies C. R.; Saravia N. G. Advances in leishmaniasis. Lancet 2005, 366, 1561-1577.
7. Silveira F. T.; Lainson R.; Corbett C. E. Clinical and immunopathological spectrum of American cutaneous leishmaniasis with special reference to the disease in Amazonian Brazil: a review. Mem. Inst. Oswaldo Cruz 2004, 99, 239-251.
8. Ilg T. Proteophosphoglycans of Leishmania. Parasitol. Today 2000, 16, 489-497.
9. Naderer T.; Vince J. E.; McConville M. J. Surface determinants of Leishmania parasites and their role in infectivity in the mammalian host. Curr. Mol. Med. 2004, 4, 649-665.
10. Descoteaux A.; Turco S. J. Glycoconjugates in Leishmania infectivity. Biochim. Biophys. Acta, Mol. Basis Dis. 1999, 1455, 341-352.
11. Sacks D. L. Leishmania-sand fly interactions controlling species-specific vector competence. Cell. Microbiol. 2001, 3, 189-196.
12. Desjardins M.; Descoteaux A. Inhibition of phagolysosomal biogenesis by the Leishmania lipophosphoglycan. J. Exp. Med. 1997, 185, 2061-2068.
13. Steinke J. W.; Platts-Mills T. A. E.; Commins S. P. The alpha-gal story: Lessons learned from connecting the dots. J. Allergy Clin. Immunol. 2015, 135, 589-596.
14. Galili U. Anti-Gal: an abundant human natural antibody of multiple pathogeneses and clinical benefits. Immunology 2013, 140, 1-11.
15. Al-Salem W. S.; Ferreira D. M.; Dyer N. A.; Alyamani E. J.; Balghonaim S. M.; Al-Mehna A. Y.; Al-Zubiany S.; Ibrahim E. K.; Al Shahrani A. M.; Alkhuailed H.; Aldahan M. A.; Al Jarallh A. M.; Abdelhady S. S.; Al-Zahrani M. H.; Almeida I. C.; Acosta-Serrano A. Detection of high levels of anti-alpha-galactosyl antibodies in sera of patients with Old World cutaneous leishmaniasis: a possible tool for diagnosis and biomarker for cure in an elimination setting. Parasitology 2014, 141, 1898-1903.
16. LaTemple D. C.; Abrams J. T.; Zhang S. Y.; Galili U. Increased immunogenicity of tumor vaccines complexed with anti-Gal: studies in knockout mice for alpha1,3galactosyltransferase. Cancer Res. 1999, 59, 3417-3423.
17. Rother R. P.; Galili U. alpha-Gal epitopes on viral glycoproteins. Subcell. Biochem. 1999, 32, 143-172.
18. Rother R. P.; Squinto S. P. The alpha-galactosyl epitope: a sugar coating that makes viruses and cells unpalatable. Cell 1996, 86, 185-188.
19. Thall A. D.; Maly P.; Lowe J. B. Oocyte Gal alpha 1,3Gal epitopes implicated in sperm adhesion to the zona pellucida glycoprotein ZP3 are not required for fertilization in the mouse. J. Biol. Chem. 1995, 270, 21437-21440.
20. Macher B. A.; Galili U. The Galalpha1,3Galbeta1,4GlcNAc-R (alpha-Gal) epitope: a carbohydrate of unique evolution and clinical relevance. Biochim. Biophys. Acta, Gen. Subj. 2008, 1780, 75-88.
21. Welsh R. M.; O'Donnell C. L.; Reed D. J.; Rother R. P. Evaluation of the Galalpha1-3Gal epitope as a host modification factor eliciting natural humoral immunity to enveloped viruses. J. Virol. 1998, 72, 4650-4656.
22. Rodriguez I. A.; Welsh R. M. Possible Role of a Cell Surface Carbohydrate in Evolution of Resistance to Viral Infections in Old World Primates. J. Virol. 2013, 87, 8317-8326.
23. Rempel B. P.; Winter H. C.; Goldstein I. J.; Hindsgaul O. Characterization of the recognition of blood group B trisaccharide derivatives by the lectin from Marasmius oreades using frontal affinity chromatography-mass spectrometry. Glycoconjugate J. 2002, 19, 175-180.
24. Winter H. C.; Mostafapour K.; Goldstein I. J. The mushroom Marasmius oreades lectin is a blood group type B agglutinin that recognizes the Galalpha 1,3Gal and Galalpha 1,3Galbeta 1,4GlcNAc porcine xenotransplantation epitopes with high affinity. J. Biol. Chem. 2002, 277, 14996-15001.
25. Ramoino P. Lectin-binding glycoconjugates in Paramecium primaurelia: changes with cellular age and starvation. Histochem. Cell Biol. 1997, 107, 321-329.
26. Wanchoo A.; Lewis M. W.; Keyhani N. O. Lectin mapping reveals stage-specific display of surface carbohydrates in in vitro and haemolymph-derived cells of the entomopathogenic fungus Beauveria bassiana. Microbiology 2009, 155, 3121-3133.
27. McCoy J. P. Jr.; Varani J.; Goldstein I. J. Enzyme-linked lectin assay (ELLA). II. Detection of carbohydrate groups on the surface of unfixed cells. Exp. Cell Res. 1984, 151, 96-103.
28. Sacks D. L.; Melby P. C., Animal models for the analysis of immune responses to leishmaniasis. Curr. Prot. Immun. 2001, Ch. 19, Unit 19.
29. Stanley A. C.; Engwerda C. R. Balancing immunity and pathology in visceral leishmaniasis. Immunol. Cell Biol. 2007, 85, 138-147.
30. Blackwell J. M.; Fakiola M.; Ibrahim M. E.; Jamieson S. E.; Jeronimo S. B.; Miller E. N.; Mishra A.; Mohamed H. S.; Peacock C. S.; Raju M.; Sundar S.; Wilson M. E. Genetics and visceral leishmaniasis: of mice and man. Parasite Immunol. 2009, 31, 254-266.
31. Loeuillet C.; Banuls A. L.; Hide M. Study of Leishmania pathogenesis in mice: experimental considerations. Parasites Vectors 2016, 9, 144.
32. Rodrigues V.; Cordeiro da Silva A.; Laforge M.; Silvestre R.; Estaquier J. Regulation of immunity during visceral Leishmania infection. Parasites Vectors 2016, 9, 118.
33. Araujo R. N.; Franco P. F.; Rodrigues H.; Santos L. C. B.; McKay C. S.; Sanhueza C. A.; Brito C. R. N.; Azevedo M. A.; Venuto A. P.; Cowan P. J.; Almeida I. C.; Finn M. G.; Marques A. F. Amblyomma sculptum tick saliva: α-Gal identification, antibody response and possible association with red meat allergy in Brazil. Int. J. Parasitol. 2016, 46, 213-220.
34. Brito C. R. N.; McKay C. S.; Azevedo M. A.; Santos L. C. B.; Nunes D. F.; D'Avila D. A.; Rodrigues de Cunha G. M.; Almeida I. C.; Gazzinelli R. T.; Galvao L. M. C.; Chiari E.; Sanhueza C. A.; Finn M. G.; Marques A. F.; Venuto A. P. Virus-like particle display of the α-Gal epitope for diagnostic assessment of Chagas disease. ACS Infect. Dis. 2016, 2, 917-922.
35. Yilmaz B.; Portugal S.; Tran T. M.; Gozzelino R.; Ramos S.; Gomes J.; Regalado A.; Cowan P. J.; d'Apice A. J.; Chong A. S.; Doumbo O. K.; Traore B.; Crompton P. D.; Silveira H.; Soares M. P. Gut microbiota elicits a protective immune response against malaria transmission. Cell 2014, 159, 1277-1289.
36. Galili U.; Wigglesworth K.; Abdel-Motal U. M. Accelerated healing of skin burns by anti-Gal/alpha-gal liposomes interaction. Burns: Journal of the International Society for Burn Injuries 2010, 36, 239-251.
37. Abdel-Motal U. M.; Wang S.; Awad A.; Lu S.; Wigglesworth K.; Galili U. Increased immunogenicity of HIV-1 p24 and gp120 following immunization with gp120/p24 fusion protein vaccine expressing alpha-gal epitopes. Vaccine 2010, 28, 1758-1765.
38. Almeida I. C.; Milani S. R.; Gorin P. A.; Travassos L. R. Complement-mediated lysis of Trypanosoma cruzi trypomastigotes by human anti-alpha-galactosyl antibodies. J. Immunol. 1991, 146, 2394-2400.
39. Fiedler J. D.; Higginson C.; Hovlid M. L.; Kislukhin A. A.; Castillejos A.; Manzenrieder F.; Campbell M. G.; Voss N. R.; Potter C. S.; Carragher B.; Finn M. G. Engineered Mutations Change the Stability and Structure of a Virus-Like Particle. Biomacromolecules 2012, 13, 2339-2348.
40. Galili U. Immune response, accommodation, and tolerance to transplantation carbohydrate antigens. Transplantation 2004, 78, 1093-1098.
41. Schonian, G.; Nasereddin, A.; Dinse, N.; Schweynoch, C.; Schallig, H. D.; Presber, W.; Jaffe, C. L., PCR diagnosis and characterization of Leishmania in local and imported clinical samples. Diag. Microbiol. Infect. Dis. 2003, 47, 349-358.
42. Galili, U., Immune response, accommodation, and tolerance to transplantation carbohydrate antigens. Transplantation 2004, 78, 1093-1098.
43. Fernandes, A. P.; Costa, M. M.; Coelho, E. A.; Michalick, M. S.; de Freitas, E.; Melo, M. N.; Luiz Tafuri, W.; Resende Dde, M.; Hermont, V.; Abrantes Cde, F.; R. T., Protective immunity against challenge with Leishmania (Leishmania chagasi) in beagle dogs vaccinated with recombinant A2 protein. Vaccine 2008, 26, 5888-5895.
44. Araujo, R. N.; Franco, P. F.; Rodrigues, H.; Santos, L. C. B.; McKay, C. S.; Sanhueza, C. A.; Brito, C. R. N.; Azevedo, M. A.; Venuto, A. P.; Cowan, P. J.; Almeida, I. C.; Finn, M. G.; Marques, A. F., Amblyomma sculptum tick saliva: α-Gal identification, antibody response and possible association with red meat allergy in Brazil. Int. J. Parasitol. 2016, 46, 213-220.
45. Fiedler, J. D.; Brown, S. D.; Lau, J.; Finn, M. G., RNA-Directed Packaging of Enzymes within Virus-Like Particles. Angew. Chem. Int. Ed. 2010, 49, 9648-9651.
46. Brito, C. R. N.; McKay, C. S.; Azevedo, M. A.; Santos, L. C. B.; Nunes, D. F.; D'Avila, D. A.; Rodrigues de Cunha, G. M.; Almeida, I. C.; Gazzinelli, R. T.; Galvao, L. M. C.; Chiari, E.; Sanhueza, C. A.; Finn, M. G.; Marques, A. F., Virus-like particle display of the α-Gal epitope for diagnostic assessment of Chagas disease. ACS Infect. Dis. 2016, 2, 917-922.
47. Hong, V.; Presolski, S. I.; Ma, C.; Finn, M. G., Analysis and Optimization of Copper-Catalyzed Azide-Alkyne Cycloaddition for Bioconjugation. Angew. Chem., Int. Ed. 2009, 48, 9879-9883.
48. Nicolas, L.; Prina, E.; Lang, T.; Milon, G., Real-time PCR for detection and quantitation of leishmania in mouse tissues. J. Clin. Microbiol. 2002, 40, 1666-1669.
49. Bretagne, S.; Durand, R.; Olivi, M.; Garin, J. F.; Sulahian, A.; Rivollet, D.; Vidaud, M.; Deniau, M., Real-time PCR as a new tool for quantifying Leishmania infantum in liver in infected mice. Clin. Diag. Lab. Immunol. 2001, 8, 828-831.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1. A vaccine composition comprising:
an immunogenic protein nanoparticle; and
at least one carbohydrate, peptide and/or peptide-based molecule, or combination thereof, associated with a Leishmania protozoan;
wherein the Leishmania protozoan is selected from the group consisting of L. mexicana, L. major, L. tropica, L. amazonensis, L. infantum, L. donovani and L. braziliensis.

2. (canceled)

3. The vaccine composition of claim 1, wherein the carbohydrate is selected from the group consisting of a Gal-α1,3-Galβ disaccharide and a Gal-α1,3-Gal-β1,4-GlcNAc trisaccharide and fragments or variants thereof.

4.-6. (canceled)

8. The vaccine composition of claim 1 further comprising a pharmaceutically acceptable carrier.

9. A vaccine composition comprising:
an immunogenic protein nanoparticle; and
at least one peptide or peptide-based molecule associated with a Leishmania protozoan;
wherein one or more of the at least one peptide or peptide-based molecule is selected from the group consisting of:
glycopeptides;
peptides of 6 to 50 amino acids in length, of sequences selected from proteins expressed by the Leishmania protozoan, including peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length;
protein domains (generally larger than 50 amino acids) and/or whole proteins expressed by the Leishmania protozoan, and combinations thereof; and
peptides of length 6-50 amino acids in length that are not found in proteins produced by Leishmania protozoans, but that have three-dimensional structures which mimic peptide or glycan structures produced by the protozoan, including peptides of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 amino acids in length.

10. The vaccine composition of claim 1, wherein the at least one carbohydrate, peptide and/or peptide-based molecule are bound to the immunogenic protein nanoparticle.

11. The vaccine composition of claim 10, wherein the at least one carbohydrate, peptide and/or peptide-based molecule are bound to the surface of the immunogenic protein nanoparticle by linkers.

12. The vaccine composition of claim 11, wherein the linkers comprise molecules with chain lengths from 1 to 100 atoms, comprising alkyl chains, aryl groups, peptides, oligo(ethylene glycol) chains, peptoids, oligo- or polyesters, oligo- or polyacrylates, and oligo- or polyamides.

13. The vaccine composition of claim 1, wherein the at least one carbohydrate, peptide and/or peptide-based molecule are present on the surface of the immunogenic protein nanoparticle.

14. The vaccine composition of claim 1, wherein the immunogenic protein nanoparticle is a virus-like particle.

15. The vaccine composition of claim 14, wherein the virus-like particle is Qβ or PP7.

16. A method of administering to a subject the vaccine of claim 1.

17. The method of claim 16, wherein the method is of inducing an immune response in the subject against a Leishmania protozoan.

18. The method of claim 17, wherein the subject is infected with a Leishmania protozoan prior to administering the vaccine of claim 1, and the vaccine induces an immune response against the Leishmania protozoan.

19. The method of claim 17, wherein the subject is not infected with a Leishmania protozoan prior to administering the vaccine of claim 1, and the vaccine induces an immune response against the Leishmania protozoan.

20.-25. (canceled)

26. The method of claim 16, wherein the method is for treating the subject infected with a L. infantum protozoan.

27. The method of claim 16 further comprising the administration of one or more of an antibiotic, an anti-fungal drug, an anti-viral drug, an anti-parasitic drug, an anti-protozoal drug, and an anti-helminthic drug.

28. A method of treating a subject infected with a Leishmania protozoal comprising administering to the subject an antibody, wherein the antibody specifically binds to one or both of:
a carbohydrate selected from the group consisting of a Gal-α1,3-Galβ disaccharide and a Gal-α1,3-Gal-β1,4-GlcNAc trisaccharide and fragments or variants thereof; and
a peptide or peptide-based molecule associated with a Leishmania protozoan.

29.-38. (canceled)

39. A method of passively immunizing a subject against a Leishmania protozoan comprising administering to the subject an antibody, wherein the antibody specifically binds to one or both of:
a carbohydrate selected from the group consisting of a Gal-α1,3-Galβ disaccharide and a Gal-α1,3-Gal-β1,4-GlcNAc trisaccharide and fragments or variants thereof; and
a peptide or peptide-based molecule associated with a Leishmania protozoan.

40.-51. (canceled)

52. The vaccine composition of claim 9 further comprising:
a carbohydrate is selected from the group consisting of a Gal-α1,3-Galβ disaccharide and a Gal-α1,3-Gal-β1,4-GlcNAc trisaccharide and fragments or variants thereof; and
a pharmaceutically acceptable carrier.
